SEQUENCE LISTING (16 sequences)

SEQ ID NO: 1 - protein, 222 aa, Artificial Sequence
  Ab0001-LCCTL-HC  Light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGALPETGG

SEQ ID NO: 2 - protein, 450 aa, Artificial Sequence
  Ab0001-LCCTL-HC  Heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 214 aa, Artificial Sequence
  Ab0001-LC-HCCT  Light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 456 aa, Artificial Sequence
  Ab0001-LC-HCCT  Heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLPETGG

SEQ ID NO: 5 - protein, 214 aa, Artificial Sequence; identical to SEQ ID NO: 3
  Ab0001-LC-HCCTL  Light chain

SEQ ID NO: 6 - protein, 458 aa, Artificial Sequence
  Ab0001-LC-HCCTL  Heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGALPETGG

SEQ ID NO: 7 - protein, 220 aa, Artificial Sequence
  Ab0001-LCCT-HC  Light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECLPETGG

SEQ ID NO: 8 - protein, 450 aa, Artificial Sequence; identical to SEQ ID NO: 2
  Ab0001-LCCT-HC  Heavy chain

SEQ ID NO: 9 - protein, 220 aa, Artificial Sequence; identical to SEQ ID NO: 7
  Ab0001-LCCT-HCCT  Light chain

SEQ ID NO: 10 - protein, 456 aa, Artificial Sequence; identical to SEQ ID NO: 4
  Ab0001-LCCT-HCCT  Heavy chain

SEQ ID NO: 11 - protein, 220 aa, Artificial Sequence; identical to SEQ ID NO: 7
  Ab0001-LCCT-HCCTL  Light chain

SEQ ID NO: 12 - protein, 458 aa, Artificial Sequence; identical to SEQ ID NO: 6
  Ab0001-LCCT-HCCTL  Heavy chain

SEQ ID NO: 13 - protein, 222 aa, Artificial Sequence; identical to SEQ ID NO: 1
  Ab0001-LCCTL-HCCT  Light chain

SEQ ID NO: 14 - protein, 456 aa, Artificial Sequence; identical to SEQ ID NO: 4
  Ab0001-LCCTL-HCCT  Heavy chain

SEQ ID NO: 15 - protein, 222 aa, Artificial Sequence; identical to SEQ ID NO: 1
  Ab0001-LCCTL-HCCTL  Light chain

SEQ ID NO: 16 - protein, 458 aa, Artificial Sequence; identical to SEQ ID NO: 6
  Ab0001-LCCTL-HCCTL  Heavy chain

(19) United States
(12) Patent Application Publication
(10) Pub. No.: US 2024/0181072 A1
(43) Pub. Date: Jun. 6, 2024

(54) Anti-HER2 Antibody-Immune Agonist Conjugate and Applications Thereof

(71) Applicants: GENEQUANTUM HEALTHCARE (SUZHOU) CO., LTD., Suzhou (CN); BRIGHTGENE BIO-MEDICAL TECHNOLOGY CO., LTD., Suzhou (CN)

(72) Inventors: Paul H. SONG, Suzhou (CN); Gang QIN, Suzhou (CN); Jiandong YUAN, Suzhou (CN); Chong LIU, Suzhou (CN)

(21) Appl. No.: 18/549,446

(22) PCT Filed: Mar. 7, 2022
(86) PCT No.: PCT/CN2022/079531

§ 371 Date: Sep. 7, 2023

(30) Foreign Application Priority Data
Mar. 8, 2021 (WO) PCT/CN2021/079611

(51) Int. Cl. A61K 47/68; A61K 31/4745; A61P 35/00; C07K 16/32

(52) U.S. Cl. CPC A61K 47/6803; A61K 31/4745; A61P 35/00; C07K 16/32

(57) ABSTRACT

The disclosure relates to a linking unit molecule for targeting molecule-drug conjugate, and the corresponding conjugate, the preparation and use thereof, and in particular relates to an anti-HER2 antibody-immune agonist conjugate (AIAC) as a novel type of cancer therapy.

### TECHNICAL FIELD

The present disclosure relates to the biopharmaceutical field, particularly to a linking unit for targeting molecule-immune agonist conjugate, and the corresponding conjugate, the preparing process and use thereof.

### BACKGROUND

Human epidermal growth factor receptor 2 (HER2) is a member of the epidermal growth factor receptor family having tyrosine kinase activity. Amplification or overexpression of HER2 occurs in approximately 15-30% of breast cancers and 10-30% of gastric/gastroesophageal cancers. HER2 overexpression has also been seen in other cancers like ovary, endometrium, bladder, lung, colon, and head and neck (Iqbal N. et al, Mol Biol Int. 2014:852748) . Although the efficacy of HER2-targeted therapies, such as HER2 directed antibody or antibody-drug conjugate (ADC), substantially improves the life expectancy of patients with HER2-positive disease, by nature, HER2-positive breast cancer is still a more aggressive form of the disease, with a poorer prognosis and worse outcomes than for patients with HER2-negative (and HR-positive) disease. Moreover, the therapeutic results have been proved disappointing in other HER2 overexpressing cancers. One of the numerous reasons for the poor outcome is that patents administered with HER2-targeting therapy become resistant. Immune escape of the tumor cells contributes to the process.

Immunotherapy is a new modality of cancer therapy that has shown great power. While immune checkpoint blockage represented by CLTA-4 and PD-1/L1 monoclonal antibody, which are basically T cell-based therapy, was approved for various cancer indications, there are also a lot of efforts exploring other mechanisms of immune system to fight against cancer. Targeting myeloid cells, majorly macrophages, DCs, has emerged as a promising direction. Activating macrophages and DCs by agonists or by macrophage checkpoint inhibitors enhances not only their capacity of phagocytosis to clear tumor cells, but also their functions of antigen presentation, which would more robustly ignite adaptive anti-tumor immunity.

The present invention provides an HER2 directed antibody-immune agonist conjugate (AIAC), a novel drug of tumor targeting immunotherapy.

### SUMMARY

In one aspect, provide is a compound of formula (I-1) or (I-2):

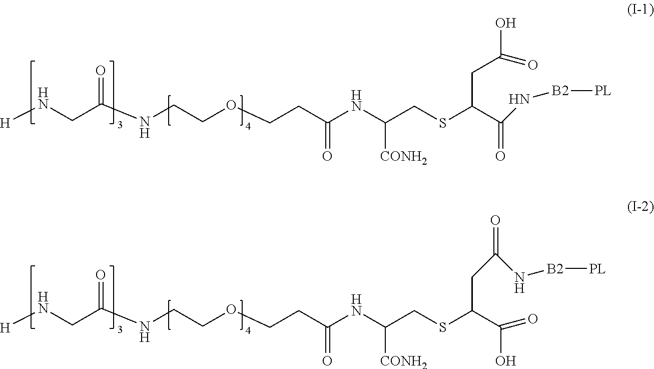

wherein B2 is —(CH₂)ₖ(CO)—NH—(C₂H₄—O)ⱼ—, k is an integer of 1 to 5, j is an integer of 1 to 3,
PL is a payload which is linked to the B2 moiety,
   preferrably, PL is Resiquimod

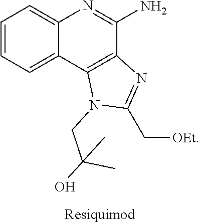 [SMILES: CCOCc1nc2c(N)nc3ccccc3c2n1CC(C)(C)O]

In yet another aspect, provided is an antibody-drug conjugate of formula (II-1) or (II-2):

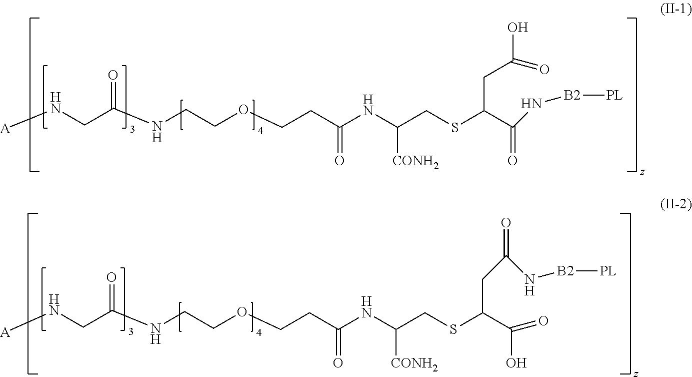

wherein B2 is —(CH₂)ₖ(CO)—NH—(C₂H₄—O)ⱼ—, k is an integer of 1 to 5, j is an integer of 1 to 3,
PL is a payload which is linked to the B2 moiety,
   preferrably, PL is Resiquimod

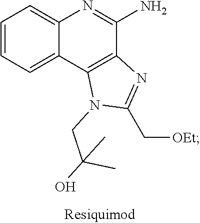 [SMILES: CCOCc1nc2c(N)nc3ccccc3c2n1CC(C)(C)O]

z is an integer of 1 to 4; preferably 1 to 2;
A is an antibody comprises a light chain having the amino acid sequence of SEQ ID NO: 1 and a heavy chain having the amino acid sequence of SEQ ID NO: 2.

The antibody-immune agonist conjugates (AIACs) of the present invention provides a novel type of tumor targeting therapy. In vitro experiments demonstrate that the AIACs can induce higher TNFα production compared to naked unmodified antibody. In vivo experiments of the AIACs show anti-tumor effect.

### BRIEF DESCRIPTION OF THE DRAWINGS

In FIGS. 7 and 8, the arrow(s) under the X-axis indicate(s) the time point of administration.

FIG. 1: Illustrative examples of the compound of formula (I′).

FIG. 2: Illustrative examples of the compound of formula (II′).

FIG. 3: Illustrative examples of the compound of formula (III′).

FIG. 4: TNFα induction activity in human PBMC-NCI N87 co-culture for conjugates AC102-5-1-1, AC102-6-1-1, and their corresponding naked unmodified antibody Ab0001 (Trastuzumab) and agonist Resiquimod.

FIG. 5: TNFα induction activity of AC102-6-1-1 and antibody in co-culture of PBMC with either NCI N87 or MDA-MB-468 cells.

FIG. 6: TNFα induction activity of AC102-9-1-1, AC102-12-1-1, AC102-10-1-1, AC102-13-1-1, and antibody in co-culture of PBMC with NCI N87 cells.

FIG. 7: Tumor volume change over time in SCID Beige mice with NCI N87 CDX model dosed with: Vehicle (PBS pH 6.5), antibody, and the test conjugate (AC102-5-1-1 or AC102-6-1-1) at 5mg/kg.

FIG. 8: Tumor volume change over time in MC38 model overexpressing hHER2 dosed with 3 mg/kg and 10 mg/kg AC102-6-1-1 and 10 mg/kg antibody.

### DETAILED DESCRIPTION

The specific embodiments are provided below to illustrate technical contents of the present disclosure. Those skilled in the art can easily understand other advantages and effects of the present disclosure through the contents disclosed in the specification. The present disclosure can also be implemented or applied through other different specific embodiments. Various modifications and variations can be made by those skilled in the art without departing from the spirit of the present disclosure.

### Definitions

Unless otherwise defined hereinafter, all technical and scientific terms used herein have the same meaning as commonly understood by those skilled in the art. The techniques used herein refer to those that are generally understood in the art, including the variants and equivalent substitutions that are obvious to those skilled in the art. While the following terms are believed to be readily comprehensible by those skilled in the art, the following definitions are set forth to better illustrate the present disclosure. When a trade name is present herein, it refers to the corresponding commodity or the active ingredient thereof. All patents, published patents applications and publications cited herein are hereby incorporated by reference.

When a certain amount, concentration, or other value or parameter is set forth in the form of a range, a preferred range, or a preferred upper limit or a preferred lower limit, it should be understood that it is equivalent to specifically revealing any range formed by combining any upper limit or preferred value with any lower limit or preferred value, regardless of whether the said range is explicitly recited. Unless otherwise stated, the numerical ranges listed herein are intended to include the endpoints of the range and all integers and fractions (decimals) within the range. For example, the expression “i is an integer of 2 to 20” means that i is any integer of 2 to 20, for example, i can be 2, 3, 4, 5, 6, 7, 8, 9, 10, 11, 12, 13, 14, 15, 16, 17, 18, 19 or 20. Other similar expressions should also be understood in a similar manner.

Unless otherwise stated herein, singular forms like “a” and “the” include the plural forms. The expression “one or more” or “at least one” may mean 1, 2, 3, 4, 5, 6, 7, 8, 9 or more.

The terms “about” and “approximately”, when used in connection with a numerical variable, generally mean that the value of the variable and all values of the variable are within experimental error (for example, within a 95% confidence interval for the mean) or within ±10% of a specified value, or a wider range.

The term “stoichiometric ratio” means matching various substances according to a certain amount by weight. For example, in the present disclosure, the active ingredient is mixed with a filler, a binder, and a lubricant in a designated weight ratio.

The term “optional” or “optionally” means the event described subsequent thereto may, but not necessarily happen, and the description includes the cases wherein the said event or circumstance happens or does not happen.

The expression “comprising” or similar expressions “including,” “containing” and “having” are open-ended, and do not exclude additional unrecited elements, steps, or ingredients. The expression “consisting of” excludes any element, step, or ingredient not designated. The expression “consisting essentially of” means that the scope is limited to the designated elements, steps or ingredients, plus elements, steps or ingredients that are optionally present that do not substantially affect the essential and novel characteristics of the claimed subject matter. It should be understood that the expression “comprising” encompasses the expressions “consisting essentially of” and “consisting of”.

The term “targeting molecule” refers to a molecule that has an affinity for a particular target (e.g., receptor, cell surface protein, cytokine, etc.). A targeting molecule can deliver the payload to a specific site in vivo through targeted delivery. A targeting molecule can recognize one or more targets. The specific target sites are defined by the targets it recognizes. For example, a targeting molecule that targets a receptor can deliver a payload to a site containing a large number of the receptor. Examples of targeting molecules include, but are not limited to antibodies, antibody fragments, binding proteins for a given antigen, antibody mimics, scaffold proteins having affinity for a given target, ligands, and the like.

As used herein, the term “antibody” is used in a broad way and particularly includes intact monoclonal antibodies, polyclonal antibodies, monospecific antibodies, multi specific antibodies (e.g., bispecific antibodies), and antibody fragments, as long as they have the desired biological activity. The antibody may be of any subtype (such as IgG, IgE, IgM, IgD, and IgA) or subclass, and may be derived from any suitable species. In some embodiments, the antibody is of human or murine origin. The antibody may also be a fully human antibody, humanized antibody or chimeric antibody prepared by recombinant methods.

Monoclonal antibodies are used herein to refer to antibodies obtained from a substantially homogeneous population of antibodies, i.e., the individual antibodies constituting the population are identical except for a small number of possible natural mutations. Monoclonal antibodies are highly specific for a single antigenic site. The word “monoclonal” refers to that the characteristics of the antibody are derived from a substantially homogeneous population of antibodies and are not to be construed as requiring some particular methods to produce the antibody.

An intact antibody or full-length antibody essentially comprises the antigen-binding variable region(s) as well as the light chain constant region(s) (C_{L} and heavy chain constant region(s) (C_{H}), which could include C_{H}1, C_{H}2, C_{H}3 and C_{H}4, depending on the subtype of the antibody. An antigen-biding variable region (also known as a fragment variable region, Fv fragment) typically comprises a light chain variable region (V_{L}) and a heavy chain variable region (V_{H}). A constant region can be a constant region with a native sequence (such as a constant region with a human native sequence) or an amino acid sequence variant thereof. The variable region recognizes and interacts with the target antigen. The constant region can be recognized by and interacts with the immune system.

An antibody fragment may comprise a portion of an intact antibody, preferably its antigen binding region or variable region. Examples of antibody fragments include Fab, Fab′, F(ab′)₂, Fd fragment consisting of V_{H} and C_{H}1 domains, Fv fragment, single-domain antibody (dAb) fragment, and isolated complementarity determining region (CDR). The Fab fragment is an antibody fragment obtained by papain digestion of a full-length immunoglobulin, or a fragment having the same structure produced by, for example, recombinant expression. A Fab fragment comprises a light chain (comprising a V_{L} and a C_{L}) and another chain, wherein the said other chain comprises a variable domain of the heavy chain (V_{H}) and a constant region domain of the heavy chain (C_{H}1). The F(ab′)₂ fragment is an antibody fragment obtained by pepsin digestion of an immunoglobulin at pH 4.0-4.5, or a fragment having the same structure produced by, for example, recombinant expression. The F(ab′)₂ fragment essentially comprises two Fab fragments, wherein each heavy chain portion comprises a few additional amino acids, including the cysteines that form disulfide bonds connecting the two fragments. A Fab′ fragment is a fragment comprising one half of a F(ab′)₂ fragment (one heavy chain and one light chain). The antibody fragment may comprise a plurality of chains joined together, for example, via a disulfide bond and/or via a peptide linking unit. Examples of antibody fragments also include single-chain Fv (scFv), Fv, dsFv, diabody, Fd and Fd′ fragments, and other fragments, including modified fragments. An antibody fragment typically comprises at least or about 50 amino acids, and typically at least or about 200 amino acids. An antigen-binding fragment can include any antibody fragment that, when inserted into an antibody framework (e.g., by substitution of the corresponding region), can result in an antibody that immunospecifically binds to the antigen.

Antibodies according to the present disclosure can be prepared using techniques well known in the art, such as the following techniques or a combination thereof: recombinant techniques, phage display techniques, synthetic techniques, or other techniques known in the art. For example, a genetically engineered recombinant antibody (or antibody mimic) can be expressed by a suitable culture system (e.g., E. coli or mammalian cells). The engineering can refer to, for example, the introduction of a ligase-specific recognition sequence at its terminals.

HER2 refers to human epidermal growth factor receptor-2, which belongs to the epidermal growth factor (EGFR) receptor tyrosine kinase family. In the present application, the terms ErbB2 and HER2 have the same meaning and can be used interchangeably.

As used herein, the term “targeting molecule-drug conjugate” is referred to as “conjugate”. Examples of conjugates include, but are not limited to, antibody-drug conjugates.

A small molecule compound refers to a molecule with a size comparable to that of an organic molecule commonly used in medicine. The term does not encompass biological macromolecules (e.g., proteins, nucleic acids, etc.), but encompasses low molecular weight peptides or derivatives thereof, such as dipeptides, tripeptides, tetrapeptides, pentapeptides, and the like. Typically, the molecular weight of the small molecule compound can be, for example, about 100 to about 2000 Da, about 200 to about 1000 Da, about 200 to about 900 Da, about 200 to about 800 Da, about 200 to about 700 Da, about 200 to about 600 Da, about 200 to about 500 Da.

Immune agonist refers to an agonist which can induce or enhance immune response to the tumor, such through activation of immune cells, including but not limited to DCs, B cells, macrophages, NK cells, and T cells. The non-limiting examples of immune agonists such as TLR agonists, including but not limited to agonists of TLR7 and/or TLR8 and/or TLR9 (e.g., Imiquimod, Resiquimod, 852A and VTX-2337) and STING agonists (e.g., ADU-S100 and MK-1454) are known in the art.

Linking unit refers to a functional group that covalently bonds two or more moieties in a compound or material. For example, the linking unit can serve to covalently bond adjuvant moieties of targeting molecule(s) and/or payload(s).

A spacer is a structure that is located between different structural modules and can

spatially separate the structural modules. The definition of spacer is not limited by whether it has a certain function or whether it can be cleaved or degraded in vivo. Examples of spacers include but are not limited to amino acids and non-amino acid structures, wherein non-amino acid structures can be, but are not limited to, amino acid derivatives or analogues. “Spacer sequence” refers to an amino acid sequence serving as a spacer, and examples thereof include but are not limited to a single amino acid such as Leu, Gln, etc., a sequence containing a plurality of amino acids, for example, a sequence containing two amino acids such as GA, etc., or, for example, GGGGS, GGGGSGGGGS, GGGGSGGGGSGGGGS, etc. Other examples of spacers include, for example, self-immolative spacers such as PABC (p-benzyloxycarbonyl), and the like.

The term “alkyl” refers to a straight or branched saturated aliphatic hydrocarbon group consisting of carbon atoms and hydrogen atoms, which is connected to the rest of the molecule through a single bond. The alkyl group may contain 1 to 20 carbon atoms, referring to C₁-C₂₀ alkyl group, for example, C₁-C₄ alkyl group, C₁-C₃ alkyl group, C₁-C₂ alkyl, C₃ alkyl, C₄ alkyl, C₃-C₆ alkyl. Non-limiting examples of alkyl groups include but are not limited to methyl, ethyl, propyl, butyl, pentyl, hexyl, isopropyl, isobutyl, sec-butyl, tert-butyl, isopentyl, 2-methylbutyl, 1-methylbutyl, 1-ethylpropyl, 1,2-dimethylpropyl, neopentyl, 1,1-dimethylpropyl, 4-methylpentyl, 3-methylpentyl, 2-methylpentyl, 1-methylpentyl, 2-ethylbutyl, 1-ethylbutyl, 3,3-dimethylbutyl, 2,2-dimethyl butyl, 1,1-dimethylbutyl, 2,3-dimethylbutyl, 1,3-dimethylbutyl or 1,2-dimethylbutyl, or their isomers. A bivalent radical refers to a group obtained from the corresponding monovalent radical by removing one hydrogen atom from a carbon atom with free valence electron(s). A bivalent radical have two connecting sites which are connected to the rest of the molecule. For example, an “alkylene” or an “alkylidene” refers to a saturated divalent hydrocarbon group, either straight or branched. Examples of alkylene groups include but are not limited to methylene (—CH₂—), ethylene (—C₂H₄—), propylene (—C₃H₆ —), butylene (—C₄H₈—), pentylene (—C₅H₁₀—), hexylene (—C₆H₁₂—), 1-methylethylene (—CH(CH₃)CH₂—), 2-methylethylene (—CH₂CH(CH₃)—), methylpropylene, ethylpropylene, and the like.

As used herein, when a group is combined with another group, the connection of the groups may be linear or branched, provided that a chemically stable structure is formed. The structure formed by such a combination can be connected to other moieties of the molecule via any suitable atom in the structure, preferably via a designated chemical bond. For example, when describing a combination of a C₁₋₄ alkylene with one of the groups including —CH₂—, —NH—, —(CO)—, —NH(CO)—, —(CO)NH—, the C₁₋₄ alkylene may form a linear connection with the above groups, such as C₁₋₄ alkylene-CH₂—, C₁₋₄ alkylene-NH—, C₁₋₄ alkylene—(CO)—, C₁₋₄ alkylene-NH(CO)—, C₁₋₄ alkylene-(CO)NH—, —CH₂—C₁₋₄ alkylene, —NH—C₁₋₄ alkylene, —(CO)—C₁₋₄ alkylene, —NH(CO)—C₁₋₄ alkylene, —(CO)NH—C₁₋₄ alkylene. The resulting bivalent structure can be further connected to other moieties of the molecule.

### Compound of Formula (I′)

In one aspect, provide is a compound of formula (I′) (formula (I′-1) or formula (I′-2) or a mixture thereof):

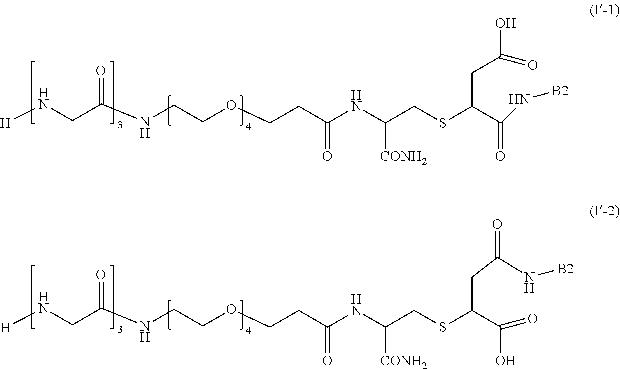

wherein,
B2 is —(CH₂)ₖ₁(CO)—NH—(C₂H₄—O)ⱼ—(CH₂)ₖ₂(CO)-Lys-R³, —(CH₂)ₖ(CO)-NH—(C₂H₄—O)ⱼ—H, or —(CH₂)ₖ(CO)—(NH—CR¹R²—(CO))_{d}—R³;
R¹ is —C₁₋₆ alkyl;
R² is selected from hydrogen and —C₁₋₆ alkyl;
R³ is a group which can leave when reacting with a group in the payload;
k , k₁, k₂ is each independently an integer of 1 to 5, j is an integer of 1 to 3, d is 1 or 2.

In one embodiment, B2 in formula (I′-1) and (I′-2) are the same.

In one embodiment, d is 1.

In one embodiment, the terminal group is hydrogen. In one embodiment, R³ is hydroxy or

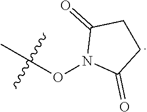 [SMILES: *ON1C(=O)CCC1=O]

In one embodiment, the terminal group R³ represents the part of structure which would not appear in the product molecule resulting from the reaction of B2 with the payload, and thus in the linking unit-payload intermediate (c.f. below) the structure moiety corresponding to B2 is the said one of or the combination of two or more of the bivalent groups. Thiosuccinimide is unstable under physiological conditions and is liable to reverse Michael addition which leads to cleavage at the conjugation site. Moreover, when another thiol compound is present in the system, thiosuccinimide may also undergo thiol exchange with the other thiol compound. Both of these reactions cause the fall-off of the payload and result in toxic side effects. In the present disclosure, the ring-opened succinimide structure

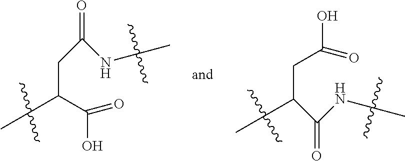 [SMILES: *NC(=O)CC(*)C(=O)O ; *NC(=O)C(*)CC(=O)O]

no longer undergoes reverse Michael addition or thiol exchange, and thus the product is more stable. Method of ring opening reaction can be found in WO2015165413A1.

### Specific Embodiment of the Formula (I′) Compound

In one embodiment, in compound of formula (I′), B2 is —(CH₂)ₖ₁(CO)—NH—(C₂H₄—O)ⱼ—(CH₂)ₖ₂(CO)-(Lys-OH), k1 is 5, j is 3, k2 is 1, Lys is connected to the rest moiety of B2 via its αamino group, and the structure of the linking unit is a mixture of the following two structures (linking unit LN102-5):

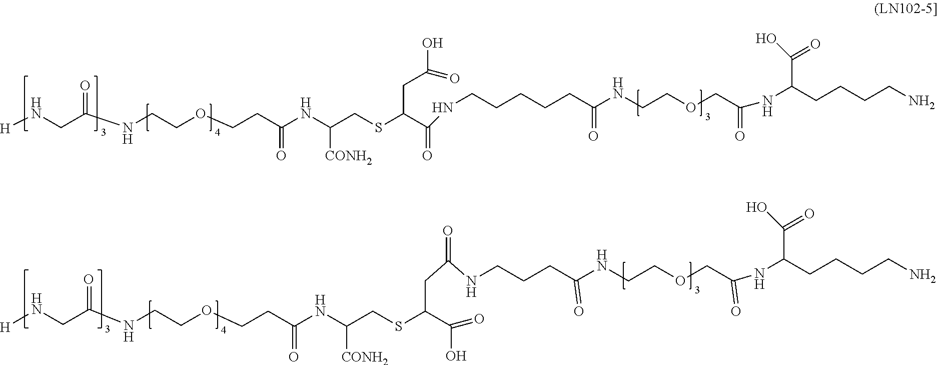

In one embodiment, in compound of formula (I′), B2 is —(CH₂)ₖ(CO)—NH—(C₂H₄—O)ⱼ—H, k is 2, j is 1, and the structure of the linking unit is a mixture of the following two structures (linking unit LN102-6):

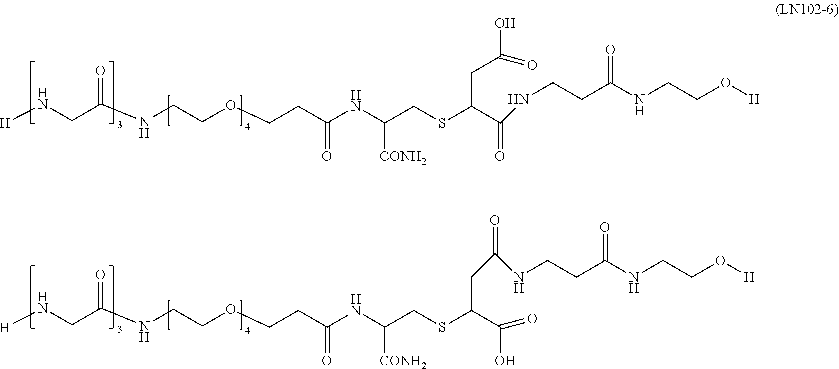

In one embodiment, in compound of formula (I′), B2 is —(CH₂)ₖ(CO)—(NH—CR¹ R²—(CO))_{d}—R³, k is 2, d is 1, R′ is (S)-methyl, R² is hydrogen, and the structure of the linking unit is a mixture of the following two structures (linking unit LN102-9):

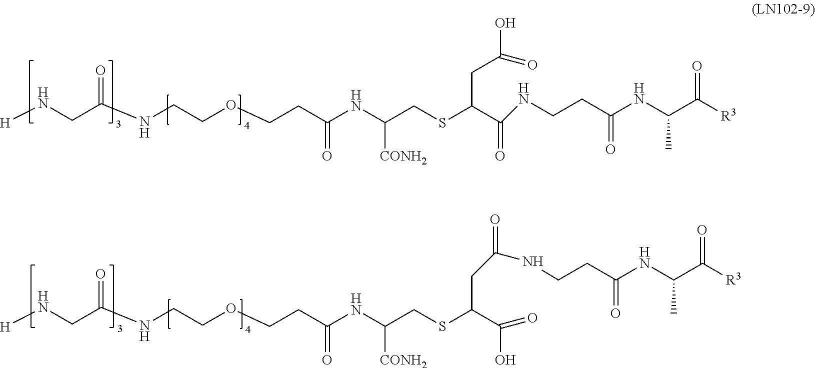

In one embodiment, in compound of formula (I′), B2 is —(CH₂)ₖ(CO)—(NH—CR¹ R²—(CO))_{d}—R³, k is 2, d is 1, R¹ is (R)-methyl, R² is hydrogen, and the structure of the linking unit is a mixture of the following two structures (linking unit LN102-10):

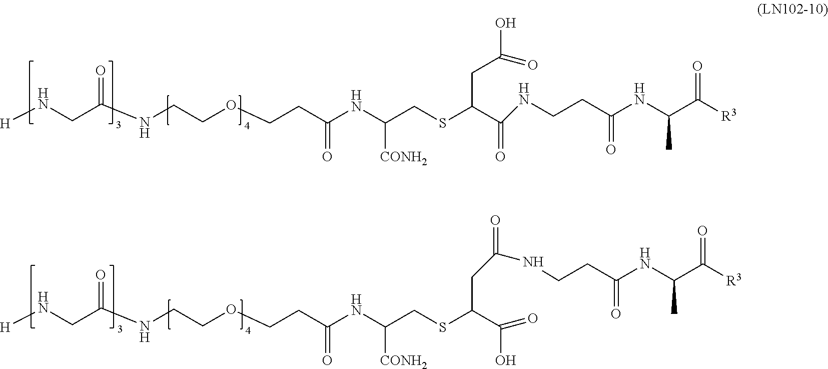

In one embodiment, in compound of formula (I′), B2 is —(CH₂)ₖ(CO)—(NH—CR¹R²—(CO))_{d}—R³, k is 2, d is 2, le and R² are methyl, R^{1′} is (S)-methyl, R^{2′} is hydrogen, and the structure of the linking unit is a mixture of the following two structures (linking unit LN102-5 12):

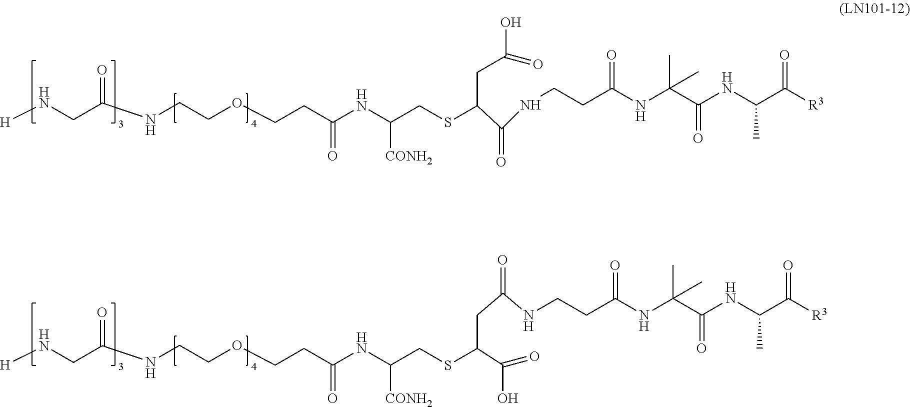

In one embodiment, in compound of formula (I′), B2 is —(CH₂)ₖ(CO)—(NH—CR¹R²—(CO))_{d}—R³, k is 2, d is 2, R¹ and R² are methyl, R^{1′} is (R)-methyl, R^{2′} is hydrogen, and the structure of the linking unit is a mixture of the following two structures (linking unit LN102-10 13):

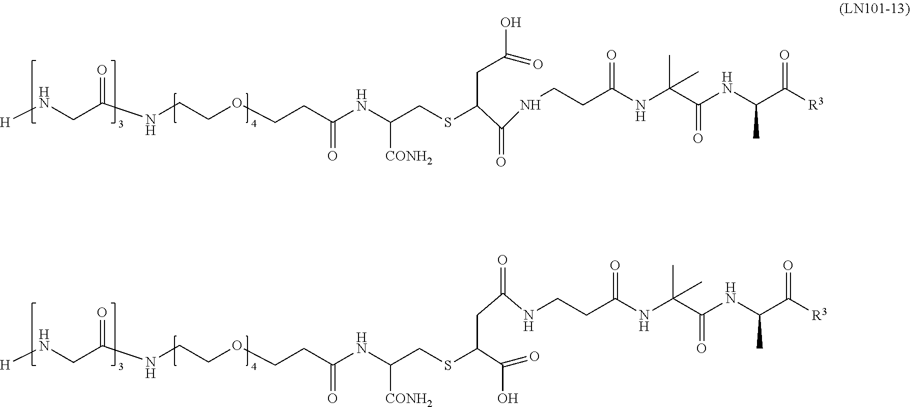

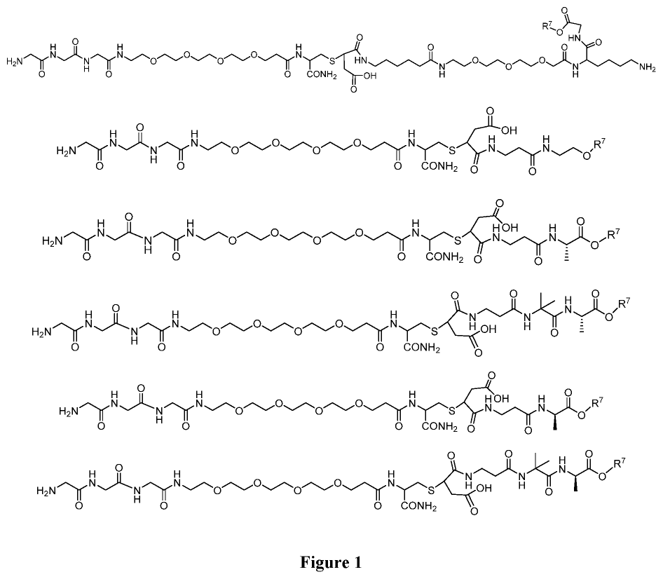

In one embodiment, the compound of formula (I′) is one of the compounds as shown in FIG. 1.

It is to be understood that when there are two or more —(CH₂)ₖC(O)— groups in the molecule, the value of each k is selected independently. In some embodiments, the “k”s in the molecule are denoted with or without additional numbers, for example k1, k2, k3, etc., wherein the numbers do not indicate any sequence, but are used merely to differentiate the “k”s. The other footnotes such as g, j, d should be understood in a similar way.

It is to be understood that when there are two or more R^{x} (x being 1, 2, 3, 4, 5, 6, 7, etc.), each R^{x} is selected independently. In some embodiments, the “x”s in the moleclue are denoted with or without additional apostrophe (') or apostrophes (such as ″, ′″, ″″, etc.) , for example R, R^{1′}, R^{1″}, R^{2′}, R^{2″}, R^{2′″}, etc. The other R^{x}s such as R³ should be understood in a similar way.

### Compound of Formula (I′) as Linking Unit

In one embodiment, the reactive group comprised by B2 can be used to covalently conjugate with a payload containing another reactive group, such that the compound of formula (I′) bears a payload.

In another embodiment, the ligase recognition sequence GGG (G is glycine) comprised by formula (I′) can be used in the conjugation by a ligase with the corresponding ligase recognition sequence LPETGG.

Thus, a compound of formula (I′) can be used as a linking unit that can be linked to a targeting molecule (such as an antibody or antigen-binding fragment thereof) and/or a payload.

One skilled in the art can synthesize the linking units by conventional solid phase or liquid phase methods.

### Payload-Bearing Formula (I′) Compound

The reactive group comprised by B2 is covalently conjugated with a payload containing another reactive group to give a payload-bearing formula (I′) compound.

In yet another aspect, provided is a compound having the structure of formula (II′-1) or (II′ -2)

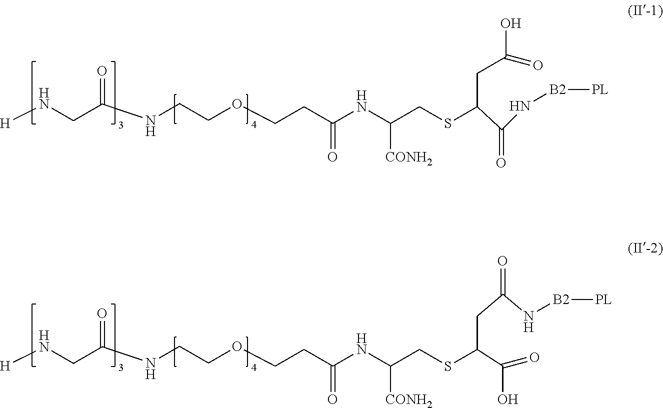

wherein
PL is a Payload which is linked to the B2 moiety of the compound of formula (I′).

### Payload

In the present disclosure, the payload may be selected from small molecule compounds, nucleic acids and analogues, tracer molecules (including fluorescent molecules, etc.), short peptides, polypeptides, peptidomimetics, and proteins. In one embodiment, the payload is selected from small molecule compounds, nucleic acid molecules, and tracer molecules. In a preferred embodiment, the payload is selected from small molecule compounds. In a more preferred embodiment, the payload is selected from cytotoxin and fragments thereof. In a more preferred embodiment, the payload is selected from immune agonist and fragments thereof.

In one embodiment, the immune agonist is selected from TLR agonists such as TLR agonists (e.g., TLR7 agonists, TLR8 agonists, TLR7/8 agonists) and STING agonists. In one embodiment, the immune agonist is selected from TLR agonists.

In one embodiment, the immune agonist is Resiquimod:

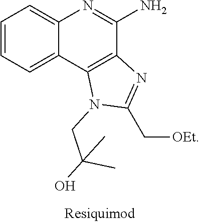 [SMILES: CCOCc1nc2c(N)nc3ccccc3c2n1CC(C)(C)O]

In one embodiment, the linking unit and the Payload are connected via reactive groups as defined above, using any reaction known in the art, including but not limit to condensation reaction, nucleophilic addition, electrophilic addition, etc.

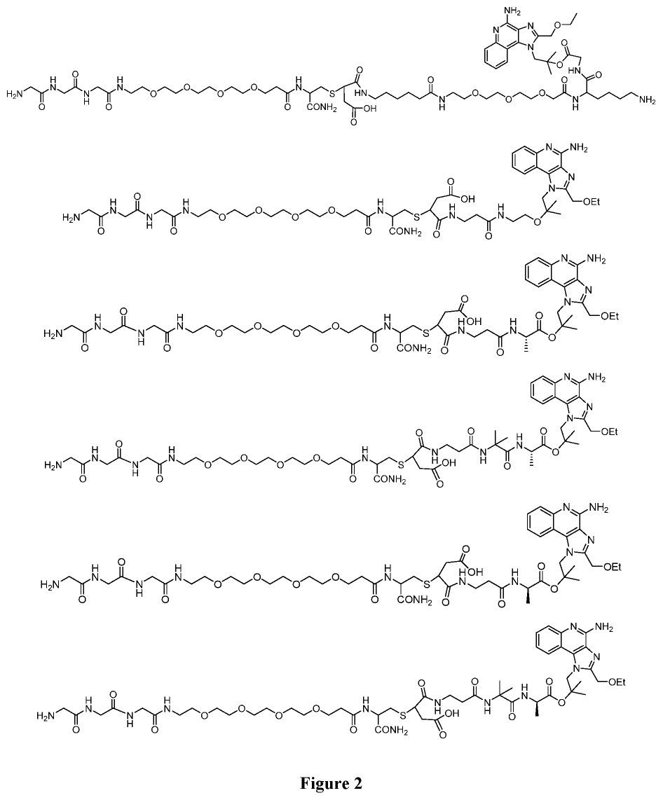

In one embodiment, the payload is an immune agonist, the antibody-immune agonist conjugate (numbered as LPx) is one of the compounds as shown in the following table and FIG. 2.

| Compound of | | |
|---|---|---|
| formula (II′) | Linking unit | PL (immune agonist) |
| LP102-4-1 | LN102-5 | Resiquimod |
| LP102-6-1 | LN102-6 | Resiquimod |
| LP102-9-1 | LN102-9 | Resiquimod |
| LP102-12-1 | LN102-12 | Resiquimod |
| LP102-10-1 | LN102-10 | Resiquimod |
| LP102-13-1 | LN102-13 | Resiquimod |

### Preparation of the Payload-Bearing Formula (I′) Compound

In one embodiment, the linking unit and the Payload are connected via reactive groups as defined above, using any reaction known in the art, including but not limit to condensation reaction, nucleophilic addition, electrophilic addition, etc.

### Compound of Formula (III′)

In one aspect, provide is a compound of formula (III′):

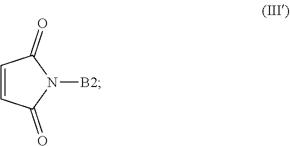

wherein B2 is as defined in formula (I′).

In one embodiment, the compound of formula (III′) could be used to prepare the payload-bearing formula (I′) compound through the following route:

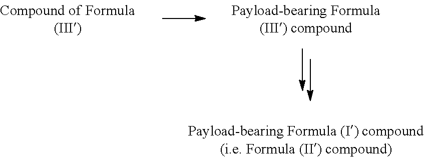

The transformation of Payload-bearing Formula (III′) compound to Payload-bearing Formula (I′) compound could be conducted using any known method in the art or as described herein. For example, single step or multi step synthesis could be conducted to introduce the structure fragment

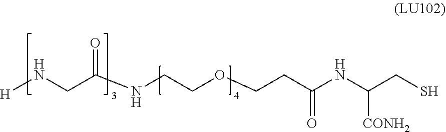

to maleimide ring in the Payload-bearing Formula (III′) compound, and then the resulting molecule which contains a succinimide moiety could undergo ring-opening reaction to open the succinimide ring and obtain the Payload-bearing Formula (I′) compound (i.e. Formula (II′) compound). In one embodiment, LU102 is introduced to the Payload-bearing Formula (III′) compound through the reaction of maleimide group contained in Formula (III′) compound with the thiol group of LU102.

### Conjugates and Preparation Thereof

In yet another aspect, provided is a conjugate having the structure of formula (IV′-1) or (IV′-2)

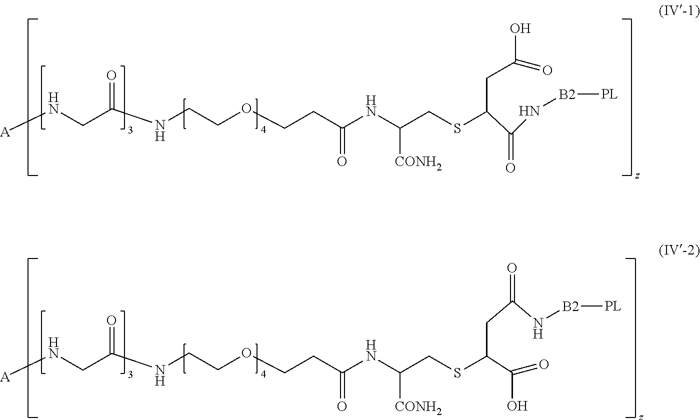

wherein,
PL is a payload which is linked to B2 moiety of the compound of formula (I′);
A is a targeting molecule which is linked to the D1 or D2 moiety of the compound of formula (I′);
z is 1 or 2.

In one embodiment, the payload is an immune agonist, which is as defined above. In one embodiment, the conjugate is an antibody-immune agonist conjugate.

### Targeting Molecule

In one embodiment, the targeting molecule is an antibody or an antigen binding fragment thereof.

In some embodiments of the present disclosure, targets recognized by the targeting molecules (such as antibodies or antigen-binding fragments thereof) include but are not limited to CD19, CD22, CD25, CD30/TNFRSF8, CD33, CD37, CD44v6, CD56, CD70, CD71, CD74, CD79b, CD117/KIT, CD123, CD138, CD142, CD174, CD227/MUC1, CD352, CLDN18.2, DLL3, ErbB2/HER2, CN33, GPNMB, ENPP3, Nectin-4, EGFRvIII, SLC44A4/AGS-5, mesothelin, CEACAMS, PSMA, TIM1, LY6E, LIV1, Nectin4, SLITRK6, HGFR/cMet, SLAMF7/CS1, EGFR, BCMA, AXL, NaPi2B, GCC, STEAP1, MUC16, Mesothelin, ETBR, EphA2, 5T4, FOLR1, LAMP1, Cadherin 6, FGFR2, FGFR3, CA6, CanAg, Integrin αV, TDGF1, Ephrin A4, Trop2, PTK7, NOTCH3, C4.4A, FLT3.

In one embodiment, the targeting molecule is an anti-human HER2 antibody or antigen binding fragment thereof. Examples of anti-human HER2 antibodies include but are not limited to Trastuzumab. Trastuzumab binds to the fourth extracellular domain (ECD4) of HER2 and is approved for the treatment of Her2-positive breast cancer and gastric cancer.

In a preferred embodiment, the anti-human HER2 antibody is one or more selected from engineered anti-HER2 antibodies based on Trastuzumab.

In a preferred embodiment, the anti-human HER2 antibody is a recombinant antibody selected from monoclonal antibody, chimeric antibody, humanized antibody, antibody fragment, and antibody mimic. In one embodiment, the antibody mimic is selected from scFv, minibody, diabody, nanobody. For the conjugation with the compound of formula (I′), the targeting molecule of the present disclosure may comprise a modified moiety to connect with the compound of formula (I′). The introduction position of such modified moiety is not limited, for example, when the targeting molecule is an antibody, its introduction position can be, but not limited to, located at the C-terminal or the N-terminal of the heavy chain or light chain of the antibody.

In one embodiment, the targeting molecule of the present disclosure is an antibody or antigen-binding fragment thereof, which may comprise terminal modification. A terminal modification refers to a modification at the C-terminal or N-terminal of the heavy chain or light chain of the antibody, which for example comprises a ligase recognition sequence. In another embodiment, the terminal modification may further comprise spacer Sp1 comprising 2-10 amino acids, wherein the antibody, Sp2 and the ligase recognition sequence are sequentially linked. In a particular embodiment, Sp2 is a spacer sequence selected from GA, GGGGS, GGGGSGGGGS, GGGGSGGGGSGGGGS, especially GA.

In a preferred embodiment, the light chain of the antibody or antigen-binding fragment thereof includes 3 types: wild-type (LC); the C-terminus modified light chain (LCCT), which is modified by direct introduction of a ligase recognition sequence LPETGG and C-terminus modified light chain (LCCTL), which is modified by introduction of short peptide spacers plus the ligase donor substrate recognition sequence LPETGG. The heavy chain of the antibody or antigen-binding fragment thereof includes 3 types: wild-type (HC); the C-terminus modified heavy chain (HCCT), which is modified by direct introduction of a ligase recognition sequence LPETGG; and C-terminus modified heavy chain (HCCTL), which is modified by introduction of short peptide spacers plus the ligase donor substrate recognition sequence LPETGG. When z in the compound of formula (IV′) is 1 or 2, the combination of the above heavy and light chains can form 8 preferred antibody molecules, see the amino acid sequence table.

The conjugates of the present disclosure can further comprise a payload. The payload is as described above.

Specific embodiments for the conjugate

In one embodiment, in formula (IV′), B2 is —(CH₂)ₖ₁(CO)—NH—(C₂H₄—O)ⱼ—(CH₂)ₖ₂(CO)-(Lys-OH)—, k1 is 5, j is 3, k2 is 1, Lys is connected to the rest moiety of B2 via its a amino group, and the structure of the conjugate is as follows (formula AC102-5):

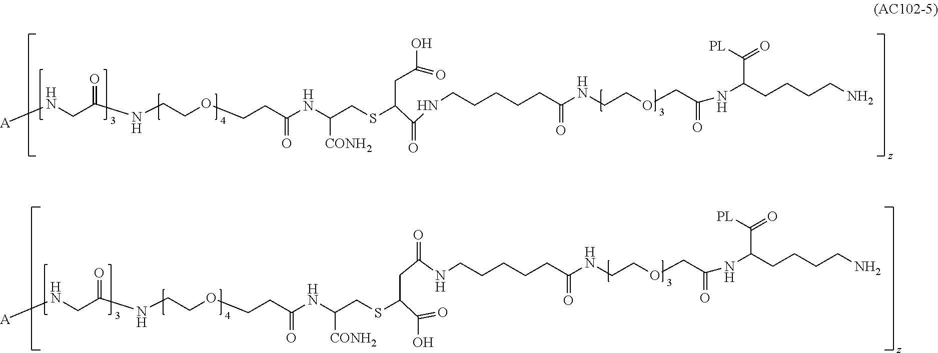

In one embodiment, in formula (IV′), B2 is —(CH₂)ₖ(CO)—NH—(C₂H₄—O)ⱼ—, k is 2, j is 1, and the structure of the conjugate is as follows (formula AC102-6):

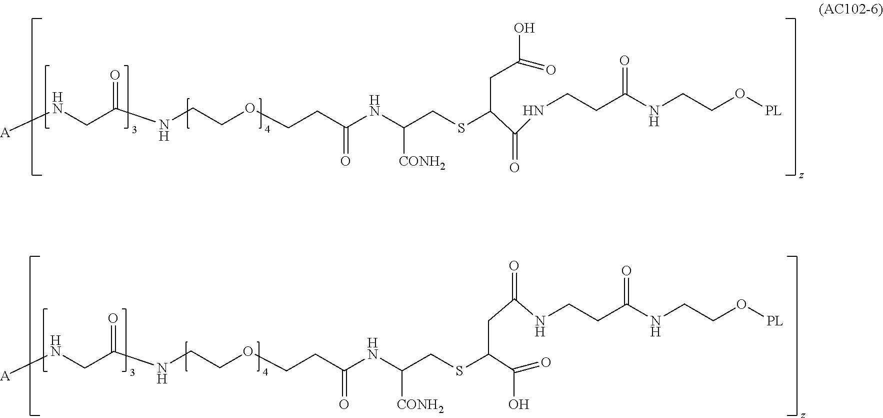

In one embodiment, in formula (IV′), B2 is —(CH₂)ₖ(CO)—(NH—CR¹ R²—(CO))_{d}—, k is 2, d is 1, R¹ is (S)-methyl, R² is hydrogen, and the structure of the conjugate is as follows (formula AC102-9):

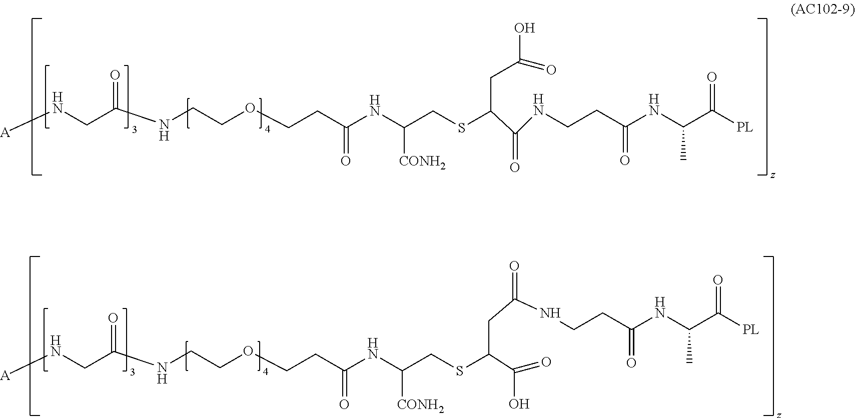

In one embodiment, in formula (IV′), B2 is —(CH₂)ₖ(CO)—(NH—CR¹ R²—(CO))_{d}—, k is 2, d is 1, R¹ is (R)-methyl, R² is hydrogen, and the structure of the conjugate is as follows (formula AC102-10):

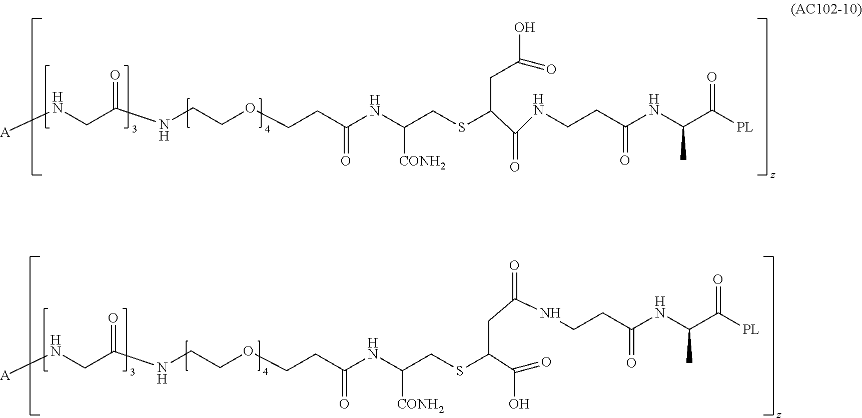

In one embodiment, in formula (IV′), B2 is —(CH₂)ₖ(CO)—(NH—CR¹ R²—(CO))_{d}—, k is 2, d is 2, R¹ and R² are methyl, R^{1′} is (S)-methyl, R^{2′} is hydrogen, and the structure of the conjugate is as follows (formula AC102-12):

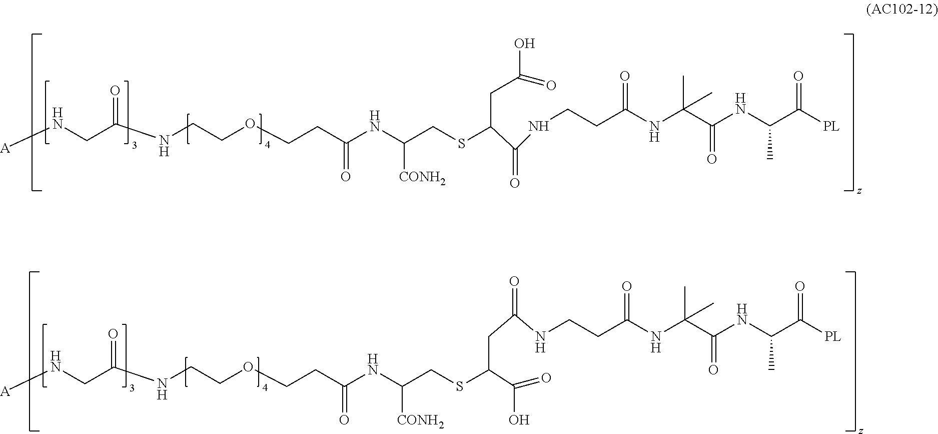

In one embodiment, in formula (IV′), B2 is —(CH₂)ₖ(CO)—(NH—CR¹ R²—(CO))_{d}—, k is 2, d is 2, R¹ and R² are methyl, R^{1″} is (R)-methyl, R^{2′} is hydrogen, and the structure of the conjugate is as follows (formula AC102-13):

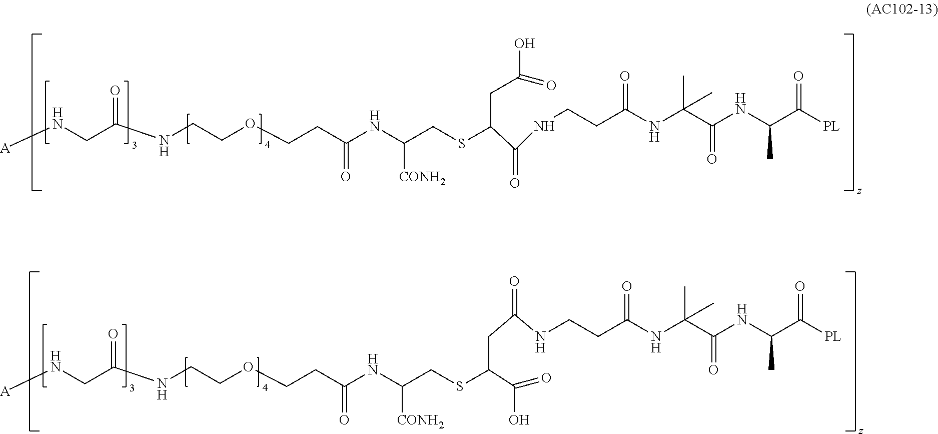

### Preparation of the Conjugate

The conjugates of the present disclosure can be prepared by any method known in the art. In some embodiments, the conjugate is prepared by the ligase-catalyzed site-specific conjugation of a targeting molecule and a payload-bearing formula (I′) compound, wherein the targeting molecule is modified by a ligase recognition sequence. The method comprises step A and step B.

### Step A. Preparation of the Linking Unit-Payload Intermediate

In a preferred embodiment, B2 in the compound of formula (I′) is covalently linked via a reactive group to a payload containing a corresponding reactive group, wherein the reactive groups are respectively as defined above.

The linking unit-payload intermediate prepared using the compound of formula (I′) of the present disclosure has defined structure, defined composition and high purity, so that when the conjugation reaction with an antibody is conducted, fewer impurities are introduced or no other impurities are introduced. When such an intermediate is used for the ligase-catalyzed site-specific conjugation with a modified antibody containing a ligase recognition sequence, a homogeneous ADC with highly controllable quality is obtained.

### Step B. Linking the Targeting Molecule to the Payload-Bearing Formula (I′) Compound

The targeting molecule of the present disclosure can be conjugated with the payload-bearing formula (I′) compound (i.e., the compound of formula (II′)) by any method known in the art. For example, ligase-catalyzed site-specific conjugation technique is applied, and the targeting molecule and the payload-bearing formula (I′) compound are linked to each other via the ligase-specific recognition sequences of the substrates. In one embodiment, the targeting molecule is an antibody with recognition sequence-based terminal modifications introduced at the C-terminal of the light chain and/or the heavy chain, and the targeting molecule is conjugated with the compound of formula (II′), under the catalysis of the wild type or optimized engineered ligase or any combination thereof, and under suitable catalytic reaction conditions.

In a specific embodiment, the ligase is Sortase A and the conjugation reaction can be represented by the following scheme:

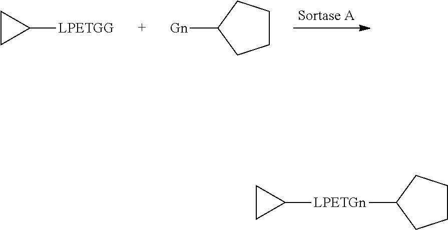

The triangle and pentagon respectively represent any of the following: a portion of an antibody or a portion of a compound of formula (II′). n is are respectively as defined above. When conjugated with Gₙ, which is the corresponding recognition sequence of the acceptor substrate, the upstream peptide bond of the glycine in the LPETGG sequence is cleaved by Sortase A, and the resulting intermediate is linked to the free N-terminal of Gₙ to generate a new peptide bond. The resulting amino acid sequence is LPETGₙ. The sequences Gₙ and LPETGG are as defined above.

### Table of Specific Conjugates

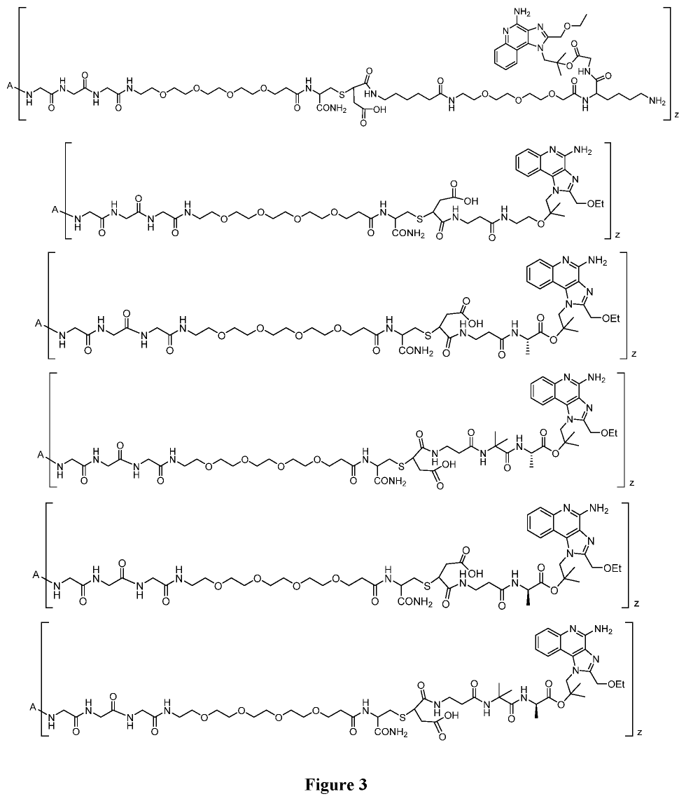

In one embodiment, the payload is an immune agonist. In one embodiment, the antibody-immune agonist conjugate is as shown in the following table and FIG. 3:

| | | PL | A |
|---|---|---|---|
| AIAC | Formula | (immune agonist) | (targeting molecule) |
| AC102-5-1-1 | AC102-5 | i-1 | Ab0001-LCCT_{L}-HC |
| AC102-6-1-1 | AC102-6 | i-1 | Ab0001-LCCT_{L}-HC |
| AC102-9-1-1 | AC102-9 | i-1 | Ab0001-LCCT_{L}-HC |
| AC102-10-1-1 | AC102-10 | i-1 | Ab0001-LCCT_{L}-HC |
| AC102-12-1-1 | AC102-12 | i-1 | Ab0001-LCCT_{L}-HC |
| AC102-13-1-1 | AC102-13 | i-1 | Ab0001-LCCT_{L}-HC |

### Pharmaceutical Composition and Pharmaceutical Preparation

Another object of the disclosure is to provide a pharmaceutical composition comprising a prophylactically or therapeutically effective amount of a conjugate of the present disclosure, and at least one pharmaceutically acceptable carrier.

The pharmaceutical composition of the present disclosure may be administered in any manner as long as it achieves the effect of preventing, alleviating, preventing or curing the symptoms of a human or animal. For example, various suitable dosage forms can be prepared according to the administration route, especially injections such as lyophilized powder for injection, injection, or sterile powder for injection.

The term “pharmaceutically acceptable” means that when contacted with tissues of the patient within the scope of normal medical judgment, no undue toxicity, irritation or allergic reaction, etc. shall arise, having reasonable advantage-disadvantage ratios and effective for the intended use.

The term pharmaceutically acceptable carrier refers to those carrier materials which are pharmaceutically acceptable and which do not interfere with the bioactivities and properties of the conjugate. Examples of aqueous carriers include but are not limited to buffered saline, and the like. The pharmaceutically acceptable carrier also includes carrier materials which brings the composition close to physiological conditions, such as pH adjusting agents, buffering agents, toxicity adjusting agents and the like, and sodium acetate, sodium chloride, potassium chloride, calcium chloride, sodium lactate, and the like.

In one embodiment, the pharmaceutical composition of the present disclosure has a drug to antibody ratio (DAR) of an integer or non-integer of 1 to 20, such as 1-10, 1-8, 1-6, 1-4, 1-3.5, 1-3, 1-2.5, preferably 1-2. In one embodiment, the pharmaceutical composition of the present disclosure has a DAR of about 1.4-about 2, preferably about 1.5-about 2, more preferably about 1.55-about 1.95. In one embodiment, the pharmaceutical composition of the present disclosure has a DAR of about 1.6-about 1.8.

### Treatment Method and Use

The conjugates of the present disclosure are useful for the treatment of tumors and/or autoimmune diseases. Tumors susceptible to conjugate treatment include those characterized by specific tumor-associated antigens or cell surface receptors, and those will be recognized by the targeting molecule in the conjugate and can be affected by the immune cell activation activity of agonist in the conjugate.

Accordingly, in yet another aspect, also provided is use of a conjugate of the present disclosure or a pharmaceutical composition of the present disclosure in the manufacture of a medicament for treating a disease, disorder or condition selected from a tumor or an autoimmune disease.

In another aspect, provided is a conjugate of the present disclosure or a pharmaceutical composition of the present disclosure for use in the treatment of a tumor or an autoimmune disease.

In a further aspect, provided is a method of treating a tumor or an autoimmune disease, the method comprising administering to an individual in need thereof an effective amount of a conjugate of the present disclosure or a pharmaceutical composition of the present disclosure

In a preferred embodiment, the conjugate of the present disclosure formed by conjugation of the anti-human HER2 antibody and the payload can specifically bind to HER2 on the surface of the tumor cell and selectively kill the HER2-expressing tumor cells. In another preferred embodiment, provided is use of a conjugate of the present disclosure or a pharmaceutical composition of the present disclosure in the manufacture of a medicament for treating a disease, disorder or condition selected from HER2-positive tumors. In a more preferred embodiment, the disease, disorder or condition is selected from breast cancer, gastric cancer, lung cancer, ovarian cancer, urothelial cancer, and the like.

The dosage of the conjugate administered to the subject can be adjusted to a considerable extent. The dosage can vary according to the particular route of administration and the needs of the subject, and can be subjected to the judgment of the health care professional.

### Beneficial Effect

The present disclosure utilizes a linking unit with unique structure and uses a ligase to catalyze the conjugation of the anti-HER2 antibody and the agonist. The conjugate of the present disclosure has good homogeneity, high activity and high selectivity. In particular, the intracellular metabolites show significantly reduced cell proliferation toxicities to the cells with low expression or no expression of target antigens. Furthermore, the toxicity of the linking unit-agonist intermediate is much lower than that of the free agonist, and thus the manufacture process of the drug is less detrimental, which is advantageous for industrial production.

The conjugate of the present disclosure achieves at least one of the following technical effects:
(1) High inhibitory activity against target cells, or strong killing effect on target cells.
(2) Good physicochemical properties (e.g., solubility, physical and/or chemical stability).
(3) Good pharmacokinetic properties (e.g., good stability in plasma, appropriate half-life and duration of action).
(4) Good safety (low toxicity on non-target normal cells or tissues, and/or fewer side effects, wider treatment window), etc.

The drug can prevent the patient from resisting to HER2-targeting therapy, and activate myeloid cells to enhance innate and adaptive immune response. It can overcome low response rate of current HER2-directed therapies.

### EXAMPLES

### Preparation Example

In order to more clearly illustrate the objects and technical solutions, the present disclosure is further described below with reference to specific examples. It is to be understood that the examples are not intended to limit the scope of the disclosure. The specific experimental methods which were not mentioned in the following examples were carried out according to conventional experimental method.

### Instruments, Materials and Reagents

Unless otherwise stated, the instruments and reagents used in the examples are commercially available. The reagents can be used directly without further purification.
MS: Thermo Fisher Q Exactive Plus, Water2795-Quattro micro triple quadrupole mass spectrometer
HPLC : Waters 2695, Agilent 1100, Agilent 1200
Semi-preparative HPLC: Lisure HP plus 50 D
Flow Cytometry: CytoFLEX S
HIC-HPLC: Butyl-HIC; mobile phase A: 25 mM PB, 2M (NH₄)₂SO₄, pH 7.0; mobile phase B: 25 mM PB, pH 7.0; flow rate: 0.8 ml/min; acquisition time: 25 min; injection amount: 20 μg; column temperature: 25° C.; detection wavelength: 280 nm; sample chamber temperature: 8° C.
SEC-HPLC: column: TSK-gel G3000 SWXL, TOSOH 7.8 mm ID×300 mm, 5 μm; mobile phase: 0.2 M KH₂PO₄, 0.25 M KCl, pH 6.2; flow rate : 0.5 ml/min; acquisition time: 30 min; injection volume: 50 μl; column temperature: 25° C.; detection wavelength; 280 nm; sample tray temperature: 8° C.
CHO was obtained from Thermo Fisher Scientific; pcDNA 3.3 was obtained from Life Technology; HEK293F was obtained from Prejin; PEIMAX transfection reagent was obtained from Polyscience; Mab Select Sure ProA was obtained from GE; Capto S ImpAct was obtained from GE; Rink-amide-MBHA-resin and dichloro resin were obtained from Nankai synthesis; HCC1954 was obtained from ATCC CAT# CRL-2338; SK-BR-3 was obtained from ATCC CAT #HTB-30; BT474 cells was obtained from ATCC CAT #HTB-20; JIMT1 cells was obtained from DSMZ CAT #ACC589; Colo205 cells was obtained from ATCC CAT #CRL-222; MC38hHER2 murine colorectal cancer cells was obtained from Biocytogen; NUGC4 human gastric cancer cells was obtained from JCRB CATOCRB0834; NCI-N87 cells (ATCC CAT #CRL-5822); MDA-MB-468 was obtained from ATCC CAT# HTB-132.

### Example 1 Construction of Antibody Expression Vector, Antibody Expression, Purification and Identification

### 1.1 Production of the Modified Anti-Human HER2 Antibody Ab0001-LCCT_{L}-HC

The expression plasmids for antibody Ab0001-LCCT_{L}-HC (light chain SEQ ID NO: 1, heavy chain: SEQ ID NO: 2) were constructed as follows. The sequence of the antibody Ab0001-LCCT_{L}-HC: based on the amino acid sequence of Trastuzumab, and GALPETGG was introduced at the C-terminal of the light chain, wherein LPETGG is the recognition sequence of the ligase donor substrate, and GA is a spacer sequence. The plasmids were transfected into CHO cells and the cell population was established and screened for a highly expressed cell population, which was cultured with reference to the culture process of Trastuzumab in a 5-10L reactor, and the supernatant was collected.

### 1.2 The Purification of Antibody Ab0001-LCCT_{L}-HC

The purification of Ab0001-LCCT_{L}-HC was carried out in a standard process using the combination of Mab Select affinity chromatography and Sepharose S cation exchange chromatography, the purified products were dissolved in the original Trastuzumab drug buffer (5mM histidine-HCl, 2% Trehalose, 0.009% Polysorbate 20, PH 6.0), and frozen in small aliquots.

### 1.3 The Quality Control of Antibody Ab0001-LCCT_{L}-HC

The purity of the above purified antibody Ab0001-LCCT_{L}-HC is 98.5% by SDS-PAGE; the content of high molecular weight polymer of the sample is less than 0.4% by SEC-HPLC; endotoxin content is less than 0.098 EU/mg.

### 1.4 Preparation of Other Modified Anti-Human Antibodies

According to a similar method, a terminal modification based on the ligase recognition sequence was introduced at the C-terminal of the light and/or heavy chain of the Trastuzumab, respectively, giving a modified antibody.

The modified anti-human HER2 antibodies based on Ab0001 (Trastuzumab) are listed in Table 1. LPETGG in the terminal modification sequence is a recognition sequence of the ligase donor substrate, and GA is a spacer sequence.

TABLE 1
Modified anti-human HER2 antibodies

| | | Sequence |
|---|---|---|
| | | introduced at |
| | Sequence | the terminal |
| Ab0001-LCCT_{L}-HC light chain | SEQ ID NO: 1 | GALPETGG |
| Ab0001-LCCT_{L}-HC heavy chain | SEQ ID NO: 2 | —* |
| Ab0001-LC-HCCT light chain | SEQ ID NO: 3 | — |
| Ab0001-LC-HCCT heavy chain | SEQ ID NO: 4 | LPETGG |
| Ab0001-LC-HCCT_{L} light chain | SEQ ID NO: 5 | — |
| Ab0001-LC-HCCT_{L} heavy chain | SEQ ID NO: 6 | GALPETGG |
| Ab0001-LCCT-HC light chain | SEQ ID NO: 7 | LPETGG |
| Ab0001-LCCT-HC heavy chain | SEQ ID NO: 8 | — |
| Ab0001-LCCT-HCCT light chain | SEQ ID NO: 9 | LPETGG |
| Ab0001-LCCT-HCCT heavy chain | SEQ ID NO: 10 | LPETGG |
| Ab0001-LCCT-HCCT_{L} light chain | SEQ ID NO: 11 | LPETGG |
| Ab0001-LCCT-HCCT_{L} heavy chain | SEQ ID NO: 12 | GALPETGG |
| Ab0001-LCCT_{L}-HCCT light chain | SEQ ID NO: 13 | GALPETGG |
| Ab0001-LCCT_{L}-HCCT heavy chain | SEQ ID NO: 14 | LPETGG |
| Ab0001-LCCT_{L}-HCCT_{L} light chain | SEQ ID NO: 15 | GALPETGG |
| Ab0001-LCCT_{L}-HCCT_{L} heavy chain | SEQ ID NO: 16 | GALPETGG |

*“—” indicates no terminal modification

### Example 2 Preparation of Intermediates

### 2.1 Preparation of the Linking Unit

### Linking Units Wherein A and Lm Present

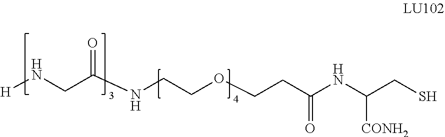

The linking unit fragment LU102 which contains moiety A of formula (I′) was synthesized by a conventional solid phase polypeptide synthesis using Rink-amide-MBHA-resin or dichloro-resin. Fmoc was used to protect the amino acid and the amino group of the Lk structure in the linking unit. The conjugation reagent was selected from HOBT, HOAt/DIC, DCC, EDCI or HATU. After synthesis, the resin was cleaved using trifluoroacetic acid. The product was purified by HPLC, lyophilized and stored for use. The linking unit fragments are listed in the following table.

TABLE

| Fragment | Sequence | Mass spectrometry |
|---|---|---|
| LU102 | GGG-NH—(C₂H₄—O)₄—C₂H₄—CO-Cys-NH₂ | 539.2 [M + H]⁺ |
| | | (Theoretical molecular weight: |
| | | 538.24) |

The linking unit fragments in the above table were reacted with a linking unit fragment which contains a maleimide structure or derivative thereof, and then underwent ring-opening reaction using the method as described in WO2015165413A1 to obtain the linking units LN102-5, LN102-6, LN102-9, LN102-10, LN102-12, LN102-13. Their structures are as shown hereinabove. in the following table:

| | Fragment | |
|---|---|---|
| | containing | |
| Linking unit | moiety A | Fragment containing moiety Lm |
| LN102-5-1 | LU102 | mc-NH—(C₂H₄—O)₃—CH₂(CO)-(Lys-OH)—OH |
| LN102-6-1 | LU102 | Mal-NH—C₂H₄—O—H |
| LN102-9-1 | LU102 | Mal-(NH—CR¹R²-(CO))—OH, |
| | | R¹ = (S)-methyl, R² = hydrogen |
| LN102-10-1 | LU102 | Mal-(NH—CR¹R²-(CO))_{d}-OH, |
| | | R¹ = (R)-methyl, R² = hydrogen |
| LN102-12-1 | LU102 | Mal-(NH—CR¹R²-(CO))_{d}—OH, k is 2, d is 2, |
| | | R¹ = R² = hydrogen, R^{1′} = (S)-methyl, R^{2′} = hydrogen |
| LN102-13-1 | LU102 | Ma-(NH—CR¹R²-(CO))_{d}—OH |
| | | R¹ = R² = hydrogen, R^{1′} = (R)-methyl, R^{2′} = hydrogen |

### 2.2 Preparation of Linking Unit-Agonist Intermediates

### 2.2.1 Preparation of Linking Unit-Agonist Intermediates LP102-6-1

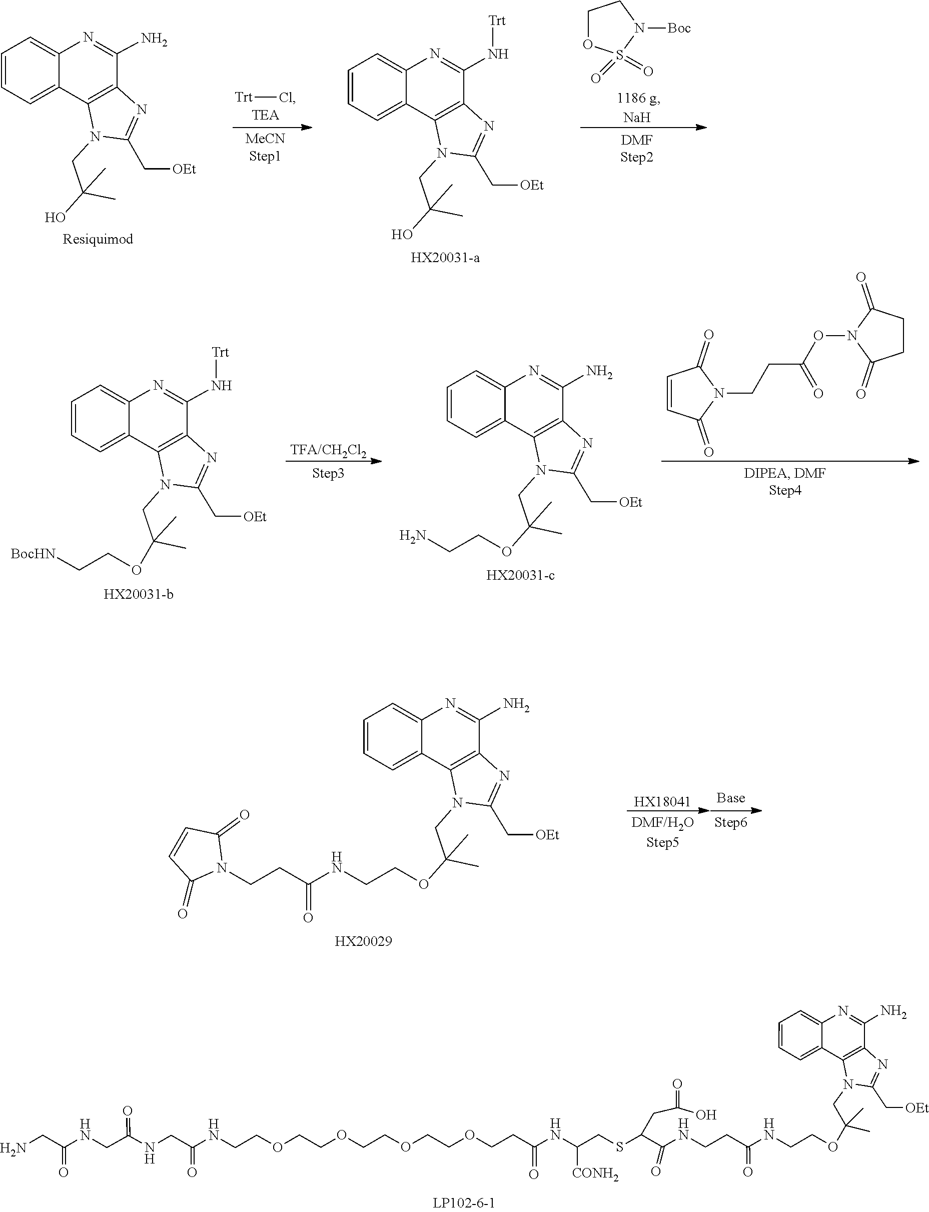

Step 1: Resiquimod (25.0 g, 79.5 mmol) was dissolved in MeCN (500 mL) and treated with Trt-Cl (33.25 g, 119.3 mmol) followed by TEA (20.12 mL, 20.12 mmol). The reaction was refluxed for 2˜3 h (TLC). The reaction mixture was concentrated in vacuo. Then the mixture was treated with AcOEt (700 mL) and with H₂O (400 mL), stirred for 30 min and separated. The organic phase was concentrated in vacuo to 300 mL and treated with n-heptane (400 mL). Then the mixture was stirred for 20 min. After filtration, the cake was beat with EtOH/H₂O (1:1, 200 mL) and filtrated. The cake was dried in vacuo to obtain target compound HX20031-a was obtained as a white solid (43.9 g, 99.1%).

Step 2: The compound (HX20031-a) (40.02 g, 72.9 mmol) was dissolved in DMF (200 mL) and cooled to 0˜10° C. NaH (60%, 3.74 g, 93.4 mmol) was added in batches. The suspension was stirred vigorously at 0˜10° C. for 1 h and then warmed to 20˜30° C. to stir for 1 h. Then the mixture was cooled to 0˜10° C. and treated with compound 1186 g (20.88 g, 93.4 mmol) in one portion. The mixture was stirred overnight at room temperature and then treated with the mixture of 10% NaH₂PO₄ (1 L) and AcOEt (1 L) slowly. The reaction mixture was stirred for 3 h and filtrated. The organic layer was concentrated and purified by silica gel column chromatography (n-heptane to n-heptane/AcOEt=10:1 to n-heptane/AcOEt=4:1) to obtain targe compound HX20031-b (24.89 g, 46.9%).

Step 3: Compound (HX20031-b) (10 g, 14.3 mmol) was treated with the mixture of TFA (40 mL) and H₂O (80 mL). The reaction mixture was stirred for 24 h at room temperature. Then the mixture was poured into MTBE (400 mL) and stirred for 2 h. After filtration, the cake was beat with MTBE (200 mL) and filtrated. The cake was dried in vacuo to obtain target compound HX20031-c was obtained as a white solid (8.51 g, 100%).

Step 4: Compound HX20031-c (6.0 g, 10.2 mmol) was dissolved in DMF (50 mL) and treated with DIPEA (3.5 mL, 20.4 mmol) and N-Succinimidyl 3-maleimidopropionate (3.27 g, 12.3 mmol). The reaction was kept at room temperature for 3 h (HPLC), then the mixture was used for next step directly.

Step 5-6: The mixture from step 4 was treated with the solution of linking unit LU102 (5.5 g, 15.3 mmol) and H₂O (50 mL). The mixture was reacted at 0-40° C. for 0.5-20 h. Then the reaction mixture was mixed with an appropriate amount of Tris Base solution or other solution that promotes the ring-opening reaction, and the reaction was performed at 0-40° C. for 0.2-20 h. After the reaction was completed, the product was purified by semi-preparative/preparative HPLC and lyophilized to obtain linking unit-agonist LP102-6-1 (3.3 g, 30% for three steps). MS m/z 1065.6 [M+H]⁺.

| Linking unit- | | | |
|---|---|---|---|
| Agonist | | | |
| intermediate | Linking unit | Agonist | Mass spectrometry |
| LP102-6-1 | LN102-6 | i-1 | Theoretical molecular |
| | | | weight: 1065.20, |
| | | | measured: [½M + H]⁺ = |
| | | | 533.6, 1065.5 [M + H]⁺ = |
| | | | 1065.5 |
| LP102-9-1 | LN102-9 | i-1 | [M + H]⁺ = 1095.1 |
| LP102-12-1 | LN102-12 | i-1 | [M + H]⁺ = 1179.2 |
| LP102-10-1 | LN102-10 | i-1 | [M + H]⁺ = 1094.3 |
| LP102-13-1 | LN102-13 | i-1 | [½M + H]⁺ = 590.4 |

### Example 3 Preparation of Targeting Molecule-Pharmaceutical Conjugates

The linking unit-agonist intermediates were respectively conjugated to an antibody in a site-specific manner by a ligase to form an AIAC. The method for conjugation reaction can be found in WO2015165413A1. The resulting AIACs are as listed in the following table:

| | Linking unit- | | |
|---|---|---|---|
| | Agonist | A | |
| AIAC | intermediate | (targeting molecule) | DAR |
| AC102-5-1-1 | LP102-5-1 | Ab0001-LCCT_{L}-HC | 1.70 |
| AC102-6-1-1 | LP102-6-1 | Ab0001-LCCT_{L}-HC | 1.74 |
| AC102-9-1-1 | LP102-9-1 | Ab0001-LCCT_{L}-HC | 1.72 |
| AC102-10-1-1 | LP102-10-1 | Ab0001-LCCT_{L}-HC | 1.74 |
| AC102-12-1-1 | LP102-12-1 | Ab0001-LCCT_{L}-HC | 1.62 |
| AC102-13-1-1 | LP102-13-1 | Ab0001-LCCT_{L}-HC | 1.63 |

### Effect Example 1 Assessment of Antibody Immune Agonist Conjugate In Vitro

### Isolation of Human Peripheral Mononuclear Cells

Human peripheral mononuclear cells were isolated from healthy blood donors using SepMate 50 and Lymphoprep (Stem Cell Technologies). The live cells was counted and the cell concentration was adjusted to 1.25×10⁶/ml in RPMI1640 medium with 10% FBS. Tumor cells were detached by trypsin, and collected. The live cells was counted and the cell concentration was adjusted to 2.5×10⁵/ml in RPMI1640 medium with 10% FBS. 12.5×10⁴ human PBMC and 2.5×10⁴ tumor cells (PBMC: tumor cells=5:1) were added into wells of 96 well plate, then the antibody or conjugate was added at indicated concentrations. The cell mixture was incubated with drugs for 18 hours, then the cell-free supernatant was collected for human TNFα ELISA.

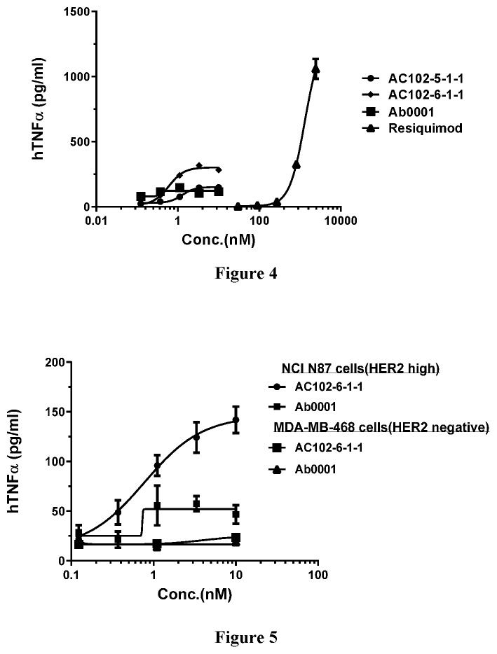

To evaluate the activity of HER2 targeted immunoconjugates, human PBMC and NCI N87 human gastric cancer cells were co-cultured at a ratio of 5:1, and the antibody or the test immunoconjugate (AC102-6-1-1 or AC102-5-1-1) at indicated concentrations were added. AC102-6-1-1 induced higher TNFα production than the antibody Ab0001, and the effective concentration of AC102-6-1-1 was much lower than the payload Resiquimod. The other immunoconjugate AC102-5-1-1 showed no obviously different activity compared to Ab0001 (FIG. 4). The activity of AC102-6-1-1 was not observed in co-culture of human PBMC and MDA-MB-468 HER2 negative cells, indicating the activity of AC102-6-1-1 was highly dependent on HER2 expression on target tumor cells (FIG. 5).

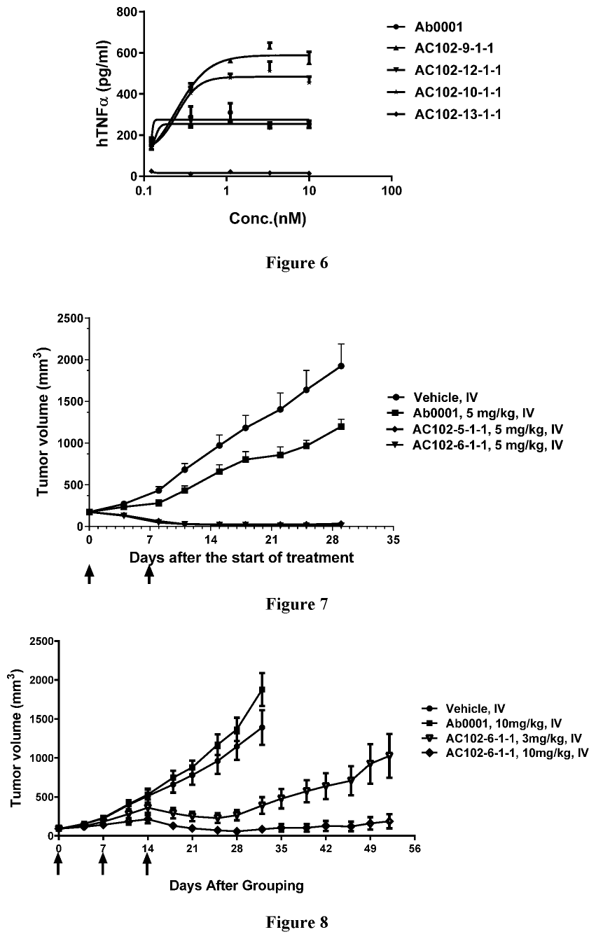

In similar experimental setting, in vitro activity of several other conjugates was evaluated (FIG. 6).

### Effect Example 2 Assessment of Antibody Immune Agonist Conjugate In Vivo

For in vivo anti-tumor efficacy study, 1×10⁷ NCI N87 human gastric cancer cells were inoculated subcutaneously in the right flank in SCID Beige mice. After 6 days, when tumor volume reached 173 mm³ on average, the tumor bearing mice were assigned and administered intravenously of Ab0001 or the test immunoconjugate (AC102-5-1-1 or AC102-6-1-1) at 5 mg/kg. The tumor volume was measured twice weekly with calipers. The antibody itself, Ab0001, showed very limited anti-tumor activity. AC102-5-1-1 and AC102-6-1-1 almost cured the tumors at the end (FIG. 7).

5×10⁵ MC38^{hHER2} murine colorectal cancer cells overexpressing human HER2 were inoculated subcutaneously in the right flank in C57BL/6 mice. After 8 days, when tumor volume reached 90 mm³ on average, the tumor bearing mice were assigned and administered intravenously of Ab0001 or AC102-6-1-1. 10 mg/kg Ab0001 showed no obvious anti-tumor activity. 3 mg/kg and 10 mg/kg AC102-6-1-1 dose-dependently inhibited tumor growth (FIG. 8).

Sequencing List

SEQ ID No. 1: Ab0001-LCCT_{L}-HC Light chain:
DIQMTQSPSSLSASVGDRVTITCRASQDVNTAVAWYQQKPGKAPKLLIYSASFLYSGVPSRFSGSRSGTD
FTLTISSLQPEDFATYYCQQHYTTPPTFGQGTKVEIKRTVAAPSVFIFPPSDEQLKSGTASVVCLLNNFYP
REAKVQWKVDNALQSGNSQESVTEQDSKDSTYSLSSTLTLSKADYEKHKVYACEVTHQGLSSPVTKSF
NRGECGALPETGG
SEQ ID No. 2: Ab0001-LCCT_{L}-HC Heavy chain:
EVQLVESGGGLVQPGGSLRLSCAASGFNIKDTYIHWVRQAPGKGLEWVARIYPTNGYTRYADSVKGRF
TISADTSKNTAYLQMNSLRAEDTAVYYCSRWGGDGFYAMDYWGQGTLVTVSSASTKGPSVFPLAPSS
KSTSGGTAALGCLVKDYFPEPVTVSWNSGALTSGVHTFPAVLQSSGLYSLSSVVTVPSSSLGTQTYICNV
NHKPSNTKVDKKVEPKSCDKTHTCPPCPAPELLGGPSVFLFPPKPKDTLMISRTPEVTCVVVDVSHEDPE
VKFNWYVDGVEVHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPPVLDSDGSFFLYS
KLTVDKSRWQQGNVFSCSVMHEALHNHYTQKSLSLSPGK
SEQ ID No. 3: Ab0001-LC-HCCT Light chain:
DIQMTQSPSSLSASVGDRVTITCRASQDVNTAVAWYQQKPGKAPKLLIYSASFLYSGVPSRFSGSRSGTD
FTLTISSLQPEDFATYYCQQHYTTPPTFGQGTKVEIKRTVAAPSVFIFPPSDEQLKSGTASVVCLLNNFYP
REAKVQWKVDNALQSGNSQESVTEQDSKDSTYSLSSTLTLSKADYEKHKVYACEVTHQGLSSPVTKSF
NRGEC
SEQ ID No. 4: Ab0001-LC-HCCT Heavy chain:
EVQLVESGGGLVQPGGSLRLSCAASGFNIKDTYIHWVRQAPGKGLEWVARIYPTNGYTRYADSVKGRF
TISADTSKNTAYLQMNSLRAEDTAVYYCSRWGGDGFYAMDYWGQGTLVTVSSASTKGPSVFPLAPSS
KSTSGGTAALGCLVKDYFPEPVTVSWNSGALTSGVHTFPAVLQSSGLYSLSSVVTVPSSSLGTQTYICNV
NHKPSNTKVDKKVEPKSCDKTHTCPPCPAPELLGGPSVFLFPPKPKDTLMISRTPEVTCVVVDVSHEDPE
VKFNWYVDGVEVHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPPVLDSDGSFFLYS
KLTVDKSRWQQGNVFSCSVMHEALHNHYTQKSLSLSPGKLPETGG
SEQ ID No. 5: Ab0001-LC-HCCT_{L} Light chain:
DIQMTQSPSSLSASVGDRVTITCRASQDVNTAVAWYQQKPGKAPKLLIYSASFLYSGVPSRFSGSRSGTD
FTLTISSLQPEDFATYYCQQHYTTPPTFGQGTKVEIKRTVAAPSVFIFPPSDEQLKSGTASVVCLLNNFYP
REAKVQWKVDNALQSGNSQESVTEQDSKDSTYSLSSTLTLSKADYEKHKVYACEVTHQGLSSPVTKSF
NRGEC
SEQ ID No. 6: Ab0001-LC-HCCT_{L} Heavy chain:
EVQLVESGGGLVQPGGSLRLSCAASGFNIKDTYIHWVRQAPGKGLEWVARIYPTNGYTRYADSVKGRF
TISADTSKNTAYLQMNSLRAEDTAVYYCSRWGGDGFYAMDYWGQGTLVTVSSASTKGPSVFPLAPSS
KSTSGGTAALGCLVKDYFPEPVTVSWNSGALTSGVHTFPAVLQSSGLYSLSSVVTVPSSSLGTQTYICNV
NHKPSNTKVDKKVEPKSCDKTHTCPPCPAPELLGGPSVFLFPPKPKDTLMISRTPEVTCVVVDVSHEDPE
VKFNWYVDGVEVHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPPVLDSDGSFFLYS
KLTVDKSRWQQGNVFSCSVMHEALHNHYTQKSLSLSPGKGALPETGG
SEQ ID No. 7: Ab0001-LCCT-HC Light chain:
DIQMTQSPSSLSASVGDRVTITCRASQDVNTAVAWYQQKPGKAPKLLIYSASFLYSGVPSRFSGSRSGTD
FTLTISSLQPEDFATYYCQQHYTTPPTFGQGTKVEIKRTVAAPSVFIFPPSDEQLKSGTASVVCLLNNFYP
REAKVQWKVDNALQSGNSQESVTEQDSKDSTYSLSSTLTLSKADYEKHKVYACEVTHQGLSSPVTKSF
NRGECLPETGG
SEQ ID No. 8: Ab0001-LCCT-HC Heavy chain:
EVQLVESGGGLVQPGGSLRLSCAASGFNIKDTYIHWVRQAPGKGLEWVARIYPTNGYTRYADSVKGRF
TISADTSKNTAYLQMNSLRAEDTAVYYCSRWGGDGFYAMDYWGQGTLVTVSSASTKGPSVFPLAPSS
KSTSGGTAALGCLVKDYFPEPVTVSWNSGALTSGVHTFPAVLQSSGLYSLSSVVTVPSSSLGTQTYICNV
NHKPSNTKVDKKVEPKSCDKTHTCPPCPAPELLGGPSVFLFPPKPKDTLMISRTPEVTCVVVDVSHEDPE
VKFNWYVDGVEVHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPPVLDSDGSFFLYS
KLTVDKSRWQQGNVFSCSVMHEALHNHYTQKSLSLSPGK
SEQ ID No. 9: Ab0001-LCCT-HCCT Light chain:
DIQMTQSPSSLSASVGDRVTITCRASQDVNTAVAWYQQKPGKAPKLLIYSASFLYSGVPSRFSGSRSGTD
FTLTISSLQPEDFATYYCQQHYTTPPTFGQGTKVEIKRTVAAPSVFIFPPSDEQLKSGTASVVCLLNNFYP
REAKVQWKVDNALQSGNSQESVTEQDSKDSTYSLSSTLTLSKADYEKHKVYACEVTHQGLSSPVTKSF
NRGECLPETGG
SEQ ID No. 10: Ab0001-LCCT-HCCT Heavy chain:
EVQLVESGGGLVQPGGSLRLSCAASGFNIKDTYIHWVRQAPGKGLEWVARIYPTNGYTRYADSVKGRF
TISADTSKNTAYLQMNSLRAEDTAVYYCSRWGGDGFYAMDYWGQGTLVTVSSASTKGPSVFPLAPSS
KSTSGGTAALGCLVKDYFPEPVTVSWNSGALTSGVHTFPAVLQSSGLYSLSSVVTVPSSSLGTQTYICNV
NHKPSNTKVDKKVEPKSCDKTHTCPPCPAPELLGGPSVFLFPPKPKDTLMISRTPEVTCVVVDVSHEDPE
VKFNWYVDGVEVHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPPVLDSDGSFFLYS
KLTVDKSRWQQGNVFSCSVMHEALHNHYTQKSLSLSPGKLPETGG
SEQ ID No. 11: Ab0001-LCCT-HCCT_{L} Light chain:
DIQMTQSPSSLSASVGDRVTITCRASQDVNTAVAWYQQKPGKAPKLLIYSASFLYSGVPSRFSGSRSGTD
FTLTISSLQPEDFATYYCQQHYTTPPTFGQGTKVEIKRTVAAPSVFIFPPSDEQLKSGTASVVCLLNNFYP
REAKVQWKVDNALQSGNSQESVTEQDSKDSTYSLSSTLTLSKADYEKHKVYACEVTHQGLSSPVTKSF
NRGECLPETGG
SEQ ID No. 12: Ab0001-LCCT-HCCT_{L} Heavy chain:
EVQLVESGGGLVQPGGSLRLSCAASGFNIKDTYIHWVRQAPGKGLEWVARIYPTNGYTRYADSVKGRF
TISADTSKNTAYLQMNSLRAEDTAVYYCSRWGGDGFYAMDYWGQGTLVTVSSASTKGPSVFPLAPSS
KSTSGGTAALGCLVKDYFPEPVTVSWNSGALTSGVHTFPAVLQSSGLYSLSSVVTVPSSSLGTQTYICNV
NHKPSNTKVDKKVEPKSCDKTHTCPPCPAPELLGGPSVFLFPPKPKDTLMISRTPEVTCVVVDVSHEDPE
VKFNWYVDGVEVHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPPVLDSDGSFFLYS
KLTVDKSRWQQGNVFSCSVMHEALHNHYTQKSLSLSPGKGALPETGG
SEQ ID No. 13: Ab0001-LCCT_{L}-HCCT Light chain:
DIQMTQSPSSLSASVGDRVTITCRASQDVNTAVAWYQQKPGKAPKLLIYSASFLYSGVPSRFSGSRSGTD
FTLTISSLQPEDFATYYCQQHYTTPPTFGQGTKVEIKRTVAAPSVFIFPPSDEQLKSGTASVVCLLNNFYP
REAKVQWKVDNALQSGNSQESVTEQDSKDSTYSLSSTLTLSKADYEKHKVYACEVTHQGLSSPVTKSF
NRGECGALPETGG
SEQ ID No. 14: Ab0001-LCCT_{L}-HCCT Heavy chain:
EVQLVESGGGLVQPGGSLRLSCAASGFNIKDTYIHWVRQAPGKGLEWVARIYPTNGYTRYADSVKGRF
TISADTSKNTAYLQMNSLRAEDTAVYYCSRWGGDGFYAMDYWGQGTLVTVSSASTKGPSVFPLAPSS
KSTSGGTAALGCLVKDYFPEPVTVSWNSGALTSGVHTFPAVLQSSGLYSLSSVVTVPSSSLGTQTYICNV
NHKPSNTKVDKKVEPKSCDKTHTCPPCPAPELLGGPSVFLFPPKPKDTLMISRTPEVTCVVVDVSHEDPE
VKFNWYVDGVEVHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPPVLDSDGSFFLYS
KLTVDKSRWQQGNVFSCSVMHEALHNHYTQKSLSLSPGKLPETGG
SEQ ID No. 15: Ab0001-LCCT_{L}-HCCT_{L} Light chain:
DIQMTQSPSSLSASVGDRVTITCRASQDVNTAVAWYQQKPGKAPKLLIYSASFLYSGVPSRFSGSRSGTD
FTLTISSLQPEDFATYYCQQHYTTPPTFGQGTKVEIKRTVAAPSVFIFPPSDEQLKSGTASVVCLLNNFYP
REAKVQWKVDNALQSGNSQESVTEQDSKDSTYSLSSTLTLSKADYEKHKVYACEVTHQGLSSPVTKSF
NRGECGALPETGG
SEQ ID No. 16: Ab0001-LCCT_{L}-HCCT_{L} Heavy chain:
EVQLVESGGGLVQPGGSLRLSCAASGFNIKDTYIHWVRQAPGKGLEWVARIYPTNGYTRYADSVKGRF
TISADTSKNTAYLQMNSLRAEDTAVYYCSRWGGDGFYAMDYWGQGTLVTVSSASTKGPSVFPLAPSS
KSTSGGTAALGCLVKDYFPEPVTVSWNSGALTSGVHTFPAVLQSSGLYSLSSVVTVPSSSLGTQTYICNV
NHKPSNTKVDKKVEPKSCDKTHTCPPCPAPELLGGPSVFLFPPKPKDTLMISRTPEVTCVVVDVSHEDPE
VKFNWYVDGVEVHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPPVLDSDGSFFLYS
KLTVDKSRWQQGNVFSCSVMHEALHNHYTQKSLSLSPGKGALPETGG

1. (canceled)

2. (canceled)

3. (canceled)

4. (canceled)

5. (canceled)

6. (canceled)

7. (canceled)

8. An antibody-drug conjugate, which is selected from the following structures: 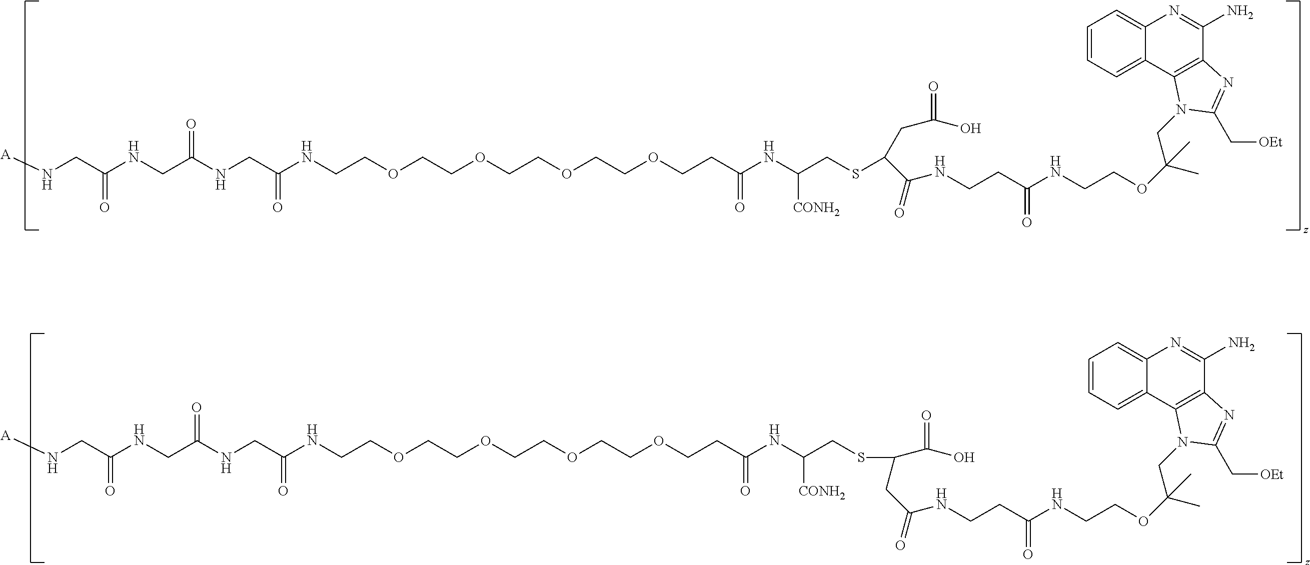
z is an integer of 1 to 4;
A is an antibody comprising a light chain having the amino acid sequence of SEQ ID NO: 1 and a heavy chain having the amino acid sequence of SEQ ID NO: 2.

9. The antibody-drug conjugate of claim 8, wherein z is 1 or 2.
